(19) United States
(12) Patent Application Publication
(10) Pub. No.: US 2024/0355469 A1
(43) Pub. Date: Oct. 24, 2024

(54) SYSTEM AND METHOD FOR PROCESSING UNSTRUCTURED DATASET CORRESPONDING TO A LUNG CANCER TECHNICAL FIELD

(71) Applicants: Rajkumar Banoth, San Antonio, TX (US); Rajendra B Dubagunta, Edison, NJ (US); Aruna Kranthi Godishala, San Antonio, TX (US); Ravinder Reddy Malga, Katy, TX (US)

(72) Inventors: Rajkumar Banoth, San Antonio, TX (US); Rajendra B Dubagunta, Edison, NJ (US); Aruna Kranthi Godishala, San Antonio, TX (US); Ravinder Reddy Malga, Katy, TX (US)

(21) Appl. No.: 18/305,413

(22) Filed: Apr. 24, 2023

(51) Int. Cl. G16H 50/20; G16H 10/60

(52) U.S. Cl. CPC G16H 50/20; G16H 10/60

(57) ABSTRACT

A system and method for processing unstructured dataset corresponding to lung cancer are disclosed. The system retrieves, from databases, unstructured dataset of individual. Further, system, extracts, from retrieved unstructured dataset, attributes associated with lung cancer of individual, and classifies unstructured dataset into lung cancer classifications, based on attributes, using Recurrent Neural Network (RNN) based model. Additionally, system generates, using chimp optimization model, stochastic population of candidate solutions comprising chimps of lung cancer classifications. Further, the system identifies optimal weight parameters for stochastic population of candidate solutions, based on oppositional-based and random-based chimp optimization model. Furthermore, the system generates, using RNN-based model, prediction result of lung cancer classifications, based on optimal weight parameters. Additionally, system validates prediction result with pre-determined prediction results and unstructured dataset of individual. Further, system outputs, on user interface of user device, prediction result for abnormalities of lung of individual, based on lung cancer classifications.

### TECHNICAL FIELD

Embodiments of the present disclosure relate to artificial intelligence systems for assisting with diagnosing medical patients and more particularly relate to a system and a method for processing unstructured datasets corresponding to lung cancer for predicting lung cancer through recurrent neural network (RNN) models using a healthcare disease diagnosis model.

### BACKGROUND

Generally, cancer is widely recognized as a genetic disease with complex and often unknown causes and mechanisms. Among the various types of cancer, lung cancer is a leading cause of death worldwide, characterized by uncontrolled growth of abnormal cells in either of the lungs. Further, there is a lack of an accepted screening instrument for an early detection of lung cancer. Advanced technologies of a radiotherapy, an intensity-modulated radiotherapy, and an image-guided radiotherapy may offer the capability of exact radiation measurement conveyance for moving articles. However, these techniques need an extra capacity to predict the exact position of the tumor against unpretentious varieties in minimum time. Machine learning techniques such as a back propagation network (BPN) and an artificial neural networks (ANN) have been utilized for classification in this field. A recurrent neural network (RNN), such as long short-term memory networks (LSTM) and a gated recurrent unit (GRU), have been successfully applied in situations with missing values, making them promising tools for handling very large real-world datasets. The LSTM model was applied to develop an algorithm that was used in cross-external validations, exploring the opportunity for increased accuracy in prediction by using time-series data.

Conventionally, the use of deep learning techniques in lung cancer detection and prediction has been provided. The conventional method provides tracking, using a recurrent neural network (RNN), internal points close to the lung tumor during radiotherapy treatment, predicting their position based on the previously computed deformation field. Another conventional method provides a multi-view convolutional recurrent neural network (MV-CRecNet), which exploits shape, size, and cross-slice variations while learning to identify lung cancer nodules from computed tomography (CT) scans.

Further another conventional method provides a method for predicting the risk of future lung cancer using image analysis and risk prediction models. The models can predict the risk of lung cancer over different periods and can be used for developing preventive therapies. Another conventional method provides plasma-based protein profiling for early-stage lung cancer prognosis. The conventional method provides biomarkers and combinations of biomarkers useful in diagnosing non-small cell lung cancer, and kits and systems for diagnosing the disease. Yet another conventional method provides a method for analyzing lung disease based on pulmonary sound. Lung sound data is acquired and processed using Mel spectrograms and cepstral coefficients to determine the presence and type of lung disease. Another conventional method provides a diagnostic inferencing with a multimodal deep memory network. The network uses different neural networks to create embeddings from medical images and electronic health records and generates patient diagnoses based on the attention given to each input.

However, the conventional methods do not use RNN with optimal weights tuning derived from a chimp optimization algorithm (ChOA) for accurate diagnosis of lung conditions, and an oppositional-based solution-generating technique that ensures the exploration of unique and opposing candidate solutions within the search area while simultaneously evaluating superior candidate solutions.

Hence, there is a need in the art for a system and a method for processing unstructured datasets corresponding to lung cancer for predicting lung cancer through recurrent neural network models using a healthcare disease diagnosis model to address at least the aforementioned issues.

### SUMMARY

This summary is provided to introduce a selection of concepts, in a simple manner, which is further described in the detailed description of the disclosure. This summary is neither intended to identify key or essential inventive concepts of the subject matter nor to determine the scope of the disclosure.

An aspect of the present disclosure provides a computer-implemented system for processing unstructured datasets corresponding to lung cancer. The system retrieves, from one or more databases, unstructured dataset corresponding to at least one individual. The unstructured dataset comprises at least one of single time-point data and time-series data. Further, the system, extracts, from the retrieved unstructured dataset, a plurality of attributes associated with a lung cancer corresponding to the at least one individual. Furthermore, the system classifies the unstructured dataset into one or more lung cancer classifications, based on the extracted plurality of attributes, using a Recurrent Neural Network (RNN) based model. Additionally, the system generates, using a chimp optimization model, a stochastic population of candidate solutions comprising one or more chimps corresponding to the one or more lung cancer classifications. Further, the system identifies one or more optimal weight parameters for the stochastic population of candidate solutions, based on an at least one of an oppositional-based chimp optimization model and a random-based chimp optimization model. One or more optimal weight parameters are identified for a convergence of the RNN-based model. Furthermore, the system generates, using the RNN-based model, a prediction result corresponding to the one or more lung cancer classifications, based on the identified one or more optimal weight parameters. Additionally, the system validates the prediction result with a pre-determined prediction results and the unstructured dataset corresponding to the at least one individual. Further, the system outputs, on a user interface associated with a user device, the validated prediction result for abnormalities of a lung of the at least one individual, based on the one or more lung cancer classifications.

Another aspect of the present disclosure provides a computer-implemented method for processing unstructured dataset corresponding to a lung cancer. The method includes retrieving, from one or more databases, unstructured dataset corresponding to at least one individual. The unstructured dataset comprises at least one of single time-point data and time-series data. Further, the method includes extracting, from the retrieved unstructured dataset, a plurality of attributes associated with a lung cancer corresponding to the at least one individual. Furthermore, the method includes classifying the unstructured dataset into one or more lung cancer classifications, based on the extracted plurality of attributes, using a Recurrent Neural Network (RNN) based model. Additionally, the method includes generating, using a chimp optimization model, a stochastic population of candidate solutions comprising one or more chimps corresponding to the one or more lung cancer classifications. Further, the method includes identifying one or more optimal weight parameters for the stochastic population of candidate solutions, based on an at least one of an oppositional-based chimp optimization model and a random-based chimp optimization model. One or more optimal weight parameters are identified for a convergence of the RNN-based model. Furthermore, the method includes generating, using the RNN-based model, a prediction result corresponding to the one or more lung cancer classifications, based on the identified one or more optimal weight parameters. Additionally, the method includes validating the prediction result with a pre-determined prediction results and the unstructured dataset corresponding to the at least one individual. Further, the method includes outputting on a user interface associated with a user device, the validated prediction result for abnormalities of the lung a lung of the at least one individual, based on the one or more lung cancer classifications.

Yet another aspect of the present disclosure provides a non-transitory computer-readable storage medium having instructions stored therein that, when executed by one or more hardware processors, cause the one or more hardware processors to retrieve, from one or more databases, unstructured dataset corresponding to at least one individual. The unstructured dataset comprises at least one of single time-point data and time-series data. Further, the processor extracts, from the retrieved unstructured dataset, a plurality of attributes associated with a lung cancer corresponding to the at least one individual. Furthermore, the processor classifies the unstructured dataset into one or more lung cancer classifications, based on the extracted plurality of attributes, using a Recurrent Neural Network (RNN) based model. Additionally, the processor generates, using a chimp optimization model, a stochastic population of candidate solutions comprising one or more chimps corresponding to the one or more lung cancer classifications. Further, the processor identifies one or more optimal weight parameters for the stochastic population of candidate solutions, based on an at least one of an oppositional-based chimp optimization model and a random-based chimp optimization model. One or more optimal weight parameters are identified for a convergence of the RNN-based model. Furthermore, the processor generates, using the RNN-based model, a prediction result corresponding to the one or more lung cancer classifications, based on the identified one or more optimal weight parameters. Additionally, the processor validates the prediction result with a pre-determined prediction results and the unstructured dataset corresponding to the at least one individual. Further, the processor outputs, on a user interface associated with a user device, the validated prediction result for abnormalities of a lung of the at least one individual, based on the one or more lung cancer classifications.

To further clarify the advantages and features of the present disclosure, a more particular description of the disclosure will follow by reference to specific embodiments thereof, which are illustrated in the appended figures. It is to be appreciated that these figures depict only typical embodiments of the disclosure and are therefore not to be considered limiting in scope. The disclosure will be described and explained with additional specificity and detail with the appended figures.

### BRIEF DESCRIPTION OF ACCOMPANYING DRAWINGS

The disclosure will be described and explained with additional specificity and detail with the accompanying figures in which:

FIG. 1 illustrates an exemplary block diagram representation of a network architecture implementing a system for processing unstructured dataset corresponding to a lung cancer, in accordance with an embodiment of the present disclosure;

FIG. 2 illustrates an exemplary block diagram representation of a computer-implemented system, such as those shown in FIG. 1, capable of processing unstructured dataset corresponding to a lung cancer, in accordance with an embodiment of the present disclosure;

FIG. 3A illustrates an exemplary architectural representation of a multi-layer perceptron (MLP) neural network architecture capable of processing unstructured dataset corresponding to a lung cancer, according to an example embodiment of the present disclosure;

FIG. 3B illustrates a flow diagram representation of a long short-term memory (LSTM) cell capable of processing unstructured dataset corresponding to a lung cancer, according to an example embodiment of the present disclosure;

FIG. 4 illustrates a flow diagram representation of an oppositional-based chimp optimization method, according to an example embodiment of the present disclosure;

FIGS. 5A and 5B illustrate graphical representations of a performance of contest techniques for different measures, according to an example embodiment of the present disclosure:

FIG. 5C illustrates a graphical representation of a converging performance of optimization techniques involved in finding optimal weights, according to an example embodiment of the present disclosure;

FIG. 6 illustrates a flow chart depicting a method for processing unstructured dataset corresponding to a lung cancer, according to an example embodiment of the present disclosure; and

FIG. 7 illustrates an exemplary block diagram representation of a hardware platform for an implementation of the disclosed system, according to an example embodiment of the present disclosure.

Further, those skilled in the art will appreciate that elements in the figures are illustrated for simplicity and may not have necessarily been drawn to scale. Furthermore, in terms of the construction of the device, one or more components of the device may have been represented in the figures by conventional symbols, and the figures may show only those specific details that are pertinent to understanding the embodiments of the present disclosure so as not to obscure the figures with details that will be readily apparent to those skilled in the art having the benefit of the description herein.

### DETAILED DESCRIPTION OF THE DISCLOSURE

For the purpose of promoting an understanding of the principles of the disclosure, reference will now be made to the embodiment illustrated in the figures and specific language will be used to describe them. It will nevertheless be understood that no limitation of the scope of the disclosure is thereby intended. Such alterations and further modifications in the illustrated system, and such further applications of the principles of the disclosure as would normally occur to those skilled in the art are to be construed as being within the scope of the present disclosure. It will be understood by those skilled in the art that the foregoing general description and the following detailed description are exemplary and explanatory of the disclosure and are not intended to be restrictive thereof.

In the present document, the word “exemplary” is used herein to mean “serving as an example, instance, or illustration.” Any embodiment or implementation of the present subject matter described herein as “exemplary” is not necessarily to be construed as preferred or advantageous over other embodiments.

The terms “comprise”, “comprising”, or any other variations thereof, are intended to cover a non-exclusive inclusion, such that one or more devices or sub-systems or elements or structures or components preceded by “comprises . . . a” does not, without more constraints, preclude the existence of other devices, sub-systems, additional sub-modules. Appearances of the phrase “in an embodiment”, “in another embodiment” and similar language throughout this specification may, but not necessarily do, all refer to the same embodiment.

Unless otherwise defined, all technical and scientific terms used herein have the same meaning as commonly understood by those skilled in the art to which this disclosure belongs. The system, methods, and examples provided herein are only illustrative and not intended to be limiting.

A computer system (standalone, client, or server computer system) configured by an application may constitute a “module” (or “subsystem”) that is configured and operated to perform certain operations. In one embodiment, the “module” or “subsystem” may be implemented mechanically or electronically, so a module includes dedicated circuitry or logic that is permanently configured (within a special-purpose processor) to perform certain operations. In another embodiment, a “module” or s “subsystem” may also comprise programmable logic or circuitry (as encompassed within a general-purpose processor or other programmable processor) that is temporarily configured by software to perform certain operations.

Accordingly, the term “module” or “subsystem” should be understood to encompass a tangible entity, be that an entity that is physically constructed permanently configured (hardwired), or temporarily configured (programmed) to operate in a certain manner and/or to perform certain operations described herein.

Embodiments of the present disclosure provide a system and a method for processing unstructured dataset corresponding to a lung cancer for predicting lung cancer through recurrent neural network (RNN) models using a healthcare disease diagnosis model. The present disclosure provides a system and method for extracting information from a historical lung cancer disease database to develop a prediction model. The proposed RNN model may be a classification-based efficient approach, in which machine learning concepts are used for the detection of the lung cancer diseases. The most effective way to reduce cancer death is to detect it earlier. The earlier detection of cancer is not an easier process but if it is detected, it is curable. The purpose of developing the RNN-based diagnosing model is to provide more accuracy than conventional methods and traditional RNN, for predicting lung cancer diseases.

The present disclosure provides an oppositional-based solution-generating technique, which ensures the examination of unique and opposite candidate solutions in the search area while evaluating the superior candidate solutions in the meanwhile. The oppositional-based solution-generating technique is validated by comparing its predicted results with the patient's prior medical record and analyzed using different performance measures. From the results, it is evident that the proposed approach has superior performance over other comparative techniques. Subsequently, for performance enhancement, the present disclosure integrates weights optimization or tuning for choosing a set of optimal weights for a learning algorithm. To resolve the computational time and complexity arising during the manual process; the present disclosure incorporates optimization techniques. The optimization technique involved in configuring the weight parameters of RNN is an oppositional-based chimp optimization algorithm (OChOA).

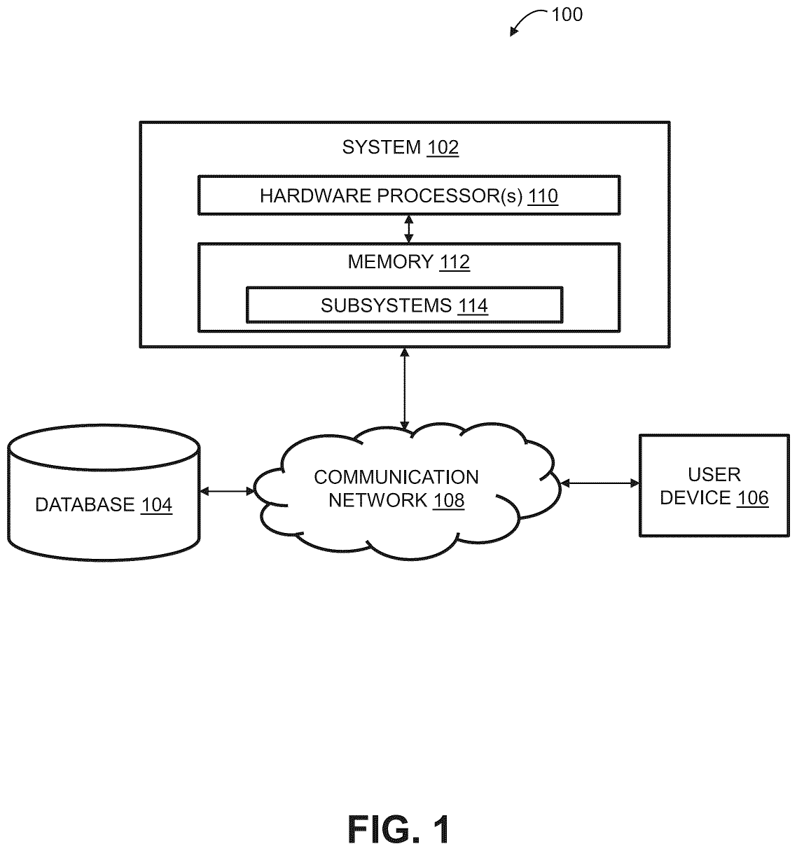

Referring now to the drawings, and more particularly to FIG. 1 through FIG. 7, where similar reference characters denote corresponding features consistently throughout the figures, there are shown preferred embodiments and these embodiments are described in the context of the following exemplary system and/or method.

FIG. 1 illustrates an exemplary block diagram representation of a network architecture 100 implementing a system 102 for processing an unstructured dataset corresponding to a lung cancer, in accordance with an embodiment of the present disclosure. According to FIG. 1, the network architecture 100 may include the system 102, a database 104, and a user device 106. The system 102 may be communicatively coupled to the database 104, and the user device 106 via a communication network 108. The communication network 108 may be a wired communication network and/or a wireless communication network. The database 104 may include, but is not limited to, unstructured dataset corresponding to at least one individual, single time-point data, time-series data, any other content, and combinations thereof.

Further, the user device 106 may be associated with, but not limited to, a user, an individual, an administrator, a vendor, a technician, a health care worker, a caretaker, a patient, a supervisor, a team, an entity, a facility, and the like. The user device 106 may be used to provide input and/or receive output to/from the system 102. The user device 106 may present to the user one or more user interfaces for the user to interact with the system 102 for the unstructured dataset processing needs. The user device 106 may be at least one of, an electrical, an electronic, an electromechanical, and a computing device. The user device 106 may include, but is not limited to, a mobile device, a smartphone, a Personal Digital Assistant (PDA), a tablet computer, a phablet computer, a wearable computing device, a Virtual Reality/Augmented Reality (VR/AR) device, a laptop, a desktop, a server, and the like. The entities and the facility may include, but are not limited to, a hospital, an e-commerce company, a merchant organization, an airline company, a hotel booking company, a company, an outlet, a manufacturing unit, an enterprise, an organization, an educational institution, a secured facility, a warehouse facility, a supply chain facility, any other facility and the like.

Further, the system 102 may be implemented by way of a single device or a combination of multiple devices that may be operatively connected or networked together. The system 102 may be implemented in hardware or a suitable combination of hardware and software. The system 102 includes a hardware processor(s) 110 and a memory 112. The memory 112 may include a plurality of subsystems 114. The system 102 may be a hardware device including the hardware processor 110 executing machine-readable program instructions for processing unstructured dataset corresponding to a lung cancer. Execution of the machine-readable program instructions by the hardware processor 110 may enable the proposed system 102 to process unstructured dataset corresponding to the lung cancer. The “hardware” may comprise a combination of discrete components, an integrated circuit, an application-specific integrated circuit, a field-programmable gate array, a digital signal processor, or other suitable hardware. The “software” may comprise one or more objects, agents, threads, lines of code, subroutines, separate software applications, two or more lines of code, or other suitable software structures operating in one or more software applications or on one or more processors.

The hardware processor 110 may include, for example, microprocessors, microcomputers, microcontrollers, digital signal processors, central processing units, state machines, logic circuits, and/or any devices that manipulate data or signals based on operational instructions. Among other capabilities, hardware processor 110 may fetch and execute computer-readable instructions in a memory operationally coupled with the system 102 for performing tasks such as data processing, input/output processing, and/or any other functions. Any reference to a task in the present disclosure may refer to an operation being or that may be performed on data.

Though few components and subsystems are disclosed in FIG. 1, there may be additional components and subsystems which is not shown, such as, but not limited to, assets, machinery, instruments, facility equipment, life safety devices, intensive care devices, treatment devices, emergency management devices, health care devices, and the like. The person skilled in the art should not be limiting the components/subsystems shown in FIG. 1. Although FIG. 1 illustrates the system 102, and the user device 106 connected to the database 104, one skilled in the art can envision that the system 102, and the user device 106 can be connected to several user devices located at different locations and several databases via the communication network 108.

Those of ordinary skilled in the art will appreciate that the hardware depicted in FIG. 1 may vary for particular implementations. For example, other peripheral devices such as an optical disk drive and the like, local area network (LAN), wide area network (WAN), wireless (e.g., wireless-fidelity (Wi-Fi)) adapter, graphics adapter, disk controller, input/output (I/O) adapter also may be used in addition or place of the hardware depicted. The depicted example is provided for explanation only and is not meant to imply architectural limitations concerning the present disclosure.

Those skilled in the art will recognize that, for simplicity and clarity, the full structure and operation of all data processing systems suitable for use with the present disclosure are not being depicted or described herein. Instead, only so much of the system 102 as is unique to the present disclosure or necessary for an understanding of the present disclosure is depicted and described. The remainder of the construction and operation of the system 102 may conform to any of the various current implementations and practices were known in the art.

In an exemplary embodiment, the system 102 may be configured to retrieve from the database(s) 104, unstructured dataset corresponding to at least one individual, The unstructured dataset may comprise, but is not limited to, single time-point data and time-series data, and the like. The individual may correspond to a patient, a healthy person, a person, a sick person, a cancer-prone patient, an early-stage cancer patient, any other person, and the like.

In an exemplary embodiment, the system 102 may be configured to extract, from the retrieved unstructured dataset, a plurality of attributes associated with a lung cancer corresponding to the at least one individual. The system 102 may extract the single time-point data and the time-series data from at least one of medical images, medical records, patient information, clinical laboratory test results, electronic health records, clinical notes, physician reports, and patient surveys, retrieved from the one or more databases.

In an exemplary embodiment, the system 102 may be configured to classify the unstructured dataset into one or more lung cancer classifications, based on the extracted plurality of attributes, using a Recurrent Neural Network (RNN) based model. The one or more lung cancer classifications may comprise, but are not limited to, an adenocarcinomas (AD) class, a squamous cell carcinomas (SQ) class, a carcinoids (COID) class, a normal lung (NL) class, and the like. In an exemplary embodiment, the system 102 may be further configured to train the RNN-based model, using the chimp optimization model and a backpropagation through time model, to classify the one or more lung cancer classes based on the time series data from the unstructured dataset.

In an exemplary embodiment, the system 102 may be configured to generate, using a chimp optimization model, a stochastic population of candidate solutions comprising one or more chimps corresponding to the one or more lung cancer classifications. In an exemplary embodiment, the system 102 may be configured to identify one or more optimal weight parameters for the stochastic population of candidate solutions, based on an at least one of an oppositional-based chimp optimization model and a random-based chimp optimization model. The one or more optimal weight parameters are identified for a convergence of the RNN-based model. For example, the weight parameters may be determined through experimentation and optimization techniques such as grid search, random search, or Bayesian optimization. It is also possible to use machine learning techniques such as neural networks to learn the optimal weight parameters. The choice of weight parameters may depend on the specific requirements and constraints of the problem being solved. For example, Genetic Algorithm (GA) may be used for identifying optimal weights for the RNN-based model.

In an exemplary embodiment, the system 102 may be configured to generate, using the RNN-based model, a prediction result corresponding to the one or more lung cancer classifications, based on the identified one or more optimal weight parameters.

In an exemplary embodiment, the system 102 may be configured to validate the prediction result with a pre-determined prediction results and the unstructured dataset corresponding to the at least one individual. In an exemplary embodiment, the system 102 may be configured to output, on a user interface associated with the user device 106, the validated prediction result for abnormalities of a lung of the at least one individual, based on the one or more lung cancer classifications.

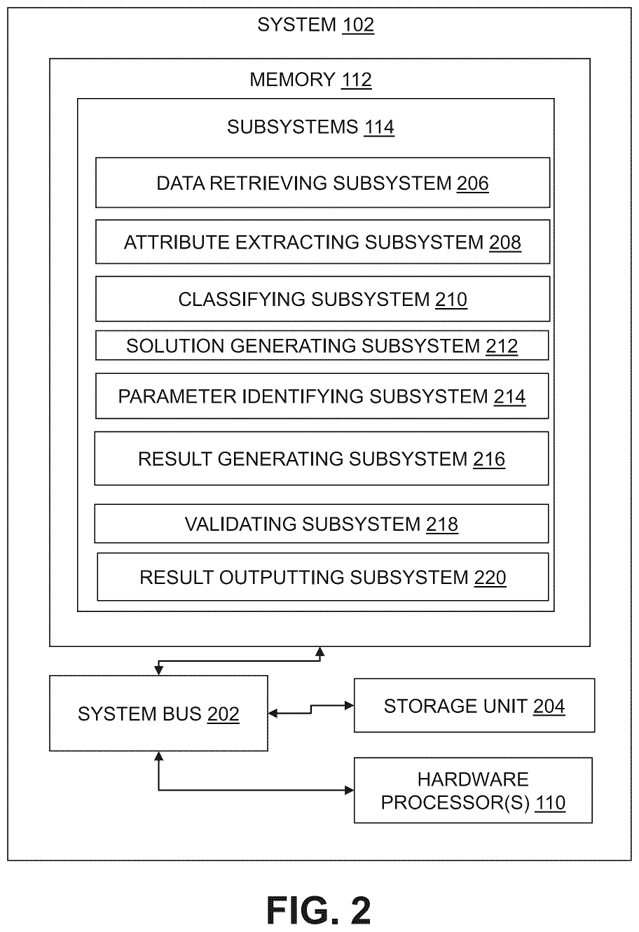

FIG. 2 illustrates an exemplary block diagram representation of a computer-implemented system 102, such as those shown in FIG. 1, capable of processing unstructured dataset corresponding to a lung cancer, in accordance with an embodiment of the present disclosure. The system 102 comprises the one or more hardware processors 110, the memory 112, and a storage unit 204. The one or more hardware processors 110, the memory 112, and the storage unit 204 are communicatively coupled through a system bus 202 or any similar mechanism. The memory 112 comprises a plurality of subsystems 114 in the form of programmable instructions executable by the one or more hardware processors 110.

Further, the plurality of subsystems 114 includes a data-retrieving subsystem 206, an attribute-extracting subsystem 208, a classifying subsystem 210, a solution-generating subsystem 212, a parameter-identifying subsystem 214, a result-generating subsystem 216, a validating subsystem 218, and a result-outputting subsystem 220.

The one or more hardware processors 110, as used herein, means any type of computational circuit, such as, but not limited to, a microprocessor unit, microcontroller, complex instruction set computing microprocessor unit, reduced instruction set computing microprocessor unit, very long instruction word microprocessor unit, explicitly parallel instruction computing microprocessor unit, graphics processing unit, digital signal processing unit, or any other type of processing circuit. The one or more hardware processors 110 may also include embedded controllers, such as generic or programmable logic devices or arrays, application-specific integrated circuits, single-chip computers, and the like.

The memory 112 may be a non-transitory volatile memory and a non-volatile memory. The memory 112 may be coupled to communicate with the one or more hardware processors 110, such as being a computer-readable storage medium. The one or more hardware processors 110 may execute machine-readable instructions and/or source code stored in the memory 112. A variety of machine-readable instructions may be stored in and accessed from the memory 112. The memory 112 may include any suitable elements for storing data and machine-readable instructions, such as read-only memory, random access memory, erasable programmable read-only memory, electrically erasable programmable read-only memory, a hard drive, a removable media drive for handling compact disks, digital video disks, diskettes, magnetic tape cartridges, memory cards, and the like. In the present embodiment, the memory 112 includes the plurality of subsystems 114 stored in the form of machine-readable instructions on any of the above-mentioned storage media and may be in communication with and executed by the one or more hardware processors 110.

The storage unit 204 may be a cloud storage or a database. The storage unit 204 may store unstructured dataset corresponding to at least one individual, single time-point data, time-series data, any other content, and combinations thereof.

In an exemplary embodiment, the data retrieving subsystem 206 may be configured to retrieve from the database(s) 104, unstructured dataset corresponding to at least one individual. The unstructured dataset may comprise, but is not limited to, single time-point data and time-series data, and the like. The individual may correspond to a patient, a healthy person, a person, a sick person, a cancer-prone patient, an early-stage cancer patient, any other person, and the like.

In an exemplary embodiment, the attribute extracting subsystem 208 may be configured to extract, from the retrieved unstructured dataset, a plurality of attributes associated with a lung cancer corresponding to the at least one individual. The system 102 may extract the single time-point data and the time-series data from at least one of medical images, medical records, patient information, clinical laboratory test results, electronic health records, clinical notes, physician reports, and patient surveys, retrieved from the one or more databases.

In an exemplary embodiment, the classifying subsystem 210 may be configured to classify the unstructured dataset into one or more lung cancer classifications, based on the extracted plurality of attributes, using a Recurrent Neural Network (RNN) based model. The one or more lung cancer classifications may comprise, but are not limited to, an adenocarcinomas (AD) class, a squamous cell carcinomas (SQ) class, a carcinoids (COID) class, and a normal lung (NL) class, and the like. In an exemplary embodiment, the system 102 may be further configured to train the RNN-based model, using the chimp optimization model and a backpropagation through time model, to classify the one or more lung cancer classes based on the time series data from the unstructured dataset.

In an exemplary embodiment, the solution-generating subsystem 212 may be configured to generate, using a chimp optimization model, a stochastic population of candidate solutions comprising one or more chimps corresponding to the one or more lung cancer classifications.

In an exemplary embodiment, the parameter identifying subsystem 214 may be configured to identify one or more optimal weight parameters for the stochastic population of candidate solutions, based on an at least one of an oppositional-based chimp optimization model and a random-based chimp optimization model. The one or more optimal weight parameters are identified for a convergence of the RNN-based model.

To identify the one or more optimal weight parameters, a parameter setting subsystem (not shown in FIG. 2) may be configured to set a time interval parameter for each iteration and a total time parameter for the chimp optimization model. Further, a value-initiating subsystem (not shown in FIG. 2) may be configured to initiate a fitness value of a candidate solution (f), a mean or average fitness value of the population of candidate solutions (m), an exploration probability (c), and an acceleration coefficient (a), for the chimp optimization model, based on the set time interval parameter and the total time parameter. Furthermore, a parameter-creating subsystem (not shown in FIG. 2) may be configured to create initial random weight parameters and generate oppositional weight parameters for the convergence of the RNN-based model. Additionally, a segregating subsystem (not shown in FIG. 2) may be configured to segregate randomly, the one or more chimps into one or more independent groups. The independent groups may comprise, but are not limited to, an attacker, a barrier, a chaser, a driver, and the like.

Further, a group-assigning subsystem (not shown in FIG. 2) may be configured to assign in each independent group, an optimal chimp from the one or more chimps. The optimal chimp may refer to the best or a most successful chimp from a group of one or more chimps. This could refer to a chimp that has the highest level of intelligence, the best physical attributes, or the most successful track record in a particular task or activity. The determination of an optimal chimp would depend on the specific criteria being evaluated and the context in which the evaluation is taking place.

Furthermore, a group extracting subsystem (not shown in FIG. 2) may be configured to extract each independent group of the assigned optimal chimp. Additionally, a value updating subsystem (not shown in FIG. 2) may be configured to update the initiated fitness value of the candidate solution (f), the mean or average fitness value of the population of candidate solutions (m), and the exploration probability (c), of each of the one or more chimps, based on a group strategy of the extracted each independent group. Moreover, a calculating subsystem (not shown in FIG. 2) may be configured to calculate the acceleration coefficient (a), and a dimensionality (d) of a search space, based on the updated fitness value of the candidate solution (f), the mean or average fitness value of the population of candidate solutions (m), and the exploration probability (c).

Additionally, a determining subsystem (not shown in FIG. 2) may be configured to determine, if a mutation rate and if a value of an inequality parameter is greater than a pre-defined value or lesser than a pre-defined value. The inequality parameter corresponds to a divergence of the candidate solutions, or a convergence toward the prey based on the value. Further, a position updating subsystem (not shown in FIG. 2) may be configured to update the position of the chimps, when the mutation rate and the value of the inequality parameter are greater than the pre-defined value. Furthermore, the position updating subsystem is configured to update the position of each chimp, and select a random search agent, when the mutation rate and the value of the inequality parameter are lesser than the pre-defined value. Further, the value updating subsystem is configured to update the fitness value of a candidate solution (f), the mean or average fitness value of the population of candidate solutions (m), the exploration probability (c), and the acceleration coefficient (a), based on the updated position of the chimps. Further, a function evaluating subsystem (not shown in FIG. 2) may be configured to evaluate the fitness function for each chimp. Moreover, a criteria-determining subsystem (not shown in FIG. 2) may be configured to determine, if the termination criteria are met for the candidate solution based on the evaluated fitness function. The termination criteria correspond to at least one of a maximum number of iterations and a desired level of fitness value. Further, a displaying subsystem (not shown in FIG. 2) is configured to display on the user interface of the user device, the optimal chimp, when the termination criteria are met, and assign optimal chimp in each group, when the termination criteria are not met.

Further, the system 102 may include an attack-triggering subsystem (not shown in FIG. 2), which may be configured to trigger the one or more chimps to attack one or more prey using a chaotic strategy comprising one or more chaotic maps. One or more prey corresponds to the candidate solutions. One or more chaotic maps update the position of the one or more chimps. One or more prey may be attacked during at least one of exploration phases and exploitation phases. The exploration phase corresponds to searching for the one or more prey by a driving operation, a blocking operation, and a chasing operation, and the exploitation phase corresponds to an attacking operation of the one or more prey. Further, a location estimating subsystem (not shown in FIG. 2) may be configured to estimate possible locations of the one or more prey by the one or more independent groups of chimps comprising the attacker, the barrier, the chaser, and the driver. Furthermore, a distance updating subsystem (not shown in FIG. 2) may be configured to update the distance of each candidate solution from the one or more prey, based on the estimated possible locations of the one or more prey. Moreover, a value decrementing subsystem (not shown in FIG. 2) may be configured to decrement a value of fitness value (f) of the candidate solution to increase an exploitation probability of attacking the one or more prey.

In an exemplary embodiment, the result-generating subsystem 216 may be configured to generate, using the RNN-based model, a prediction results corresponding to the one or more lung cancer classifications, based on the identified one or more optimal weight parameters. In an exemplary embodiment, the validating subsystem 218 may be configured to validate the prediction result with a pre-determined prediction results and the unstructured dataset corresponding to the at least one individual. In an exemplary embodiment, the result outputting subsystem 220 may be configured to output, on a user interface associated with the user device 106, the validated prediction result for abnormalities of a lung of the at least one individual, based on the one or more lung cancer classifications.

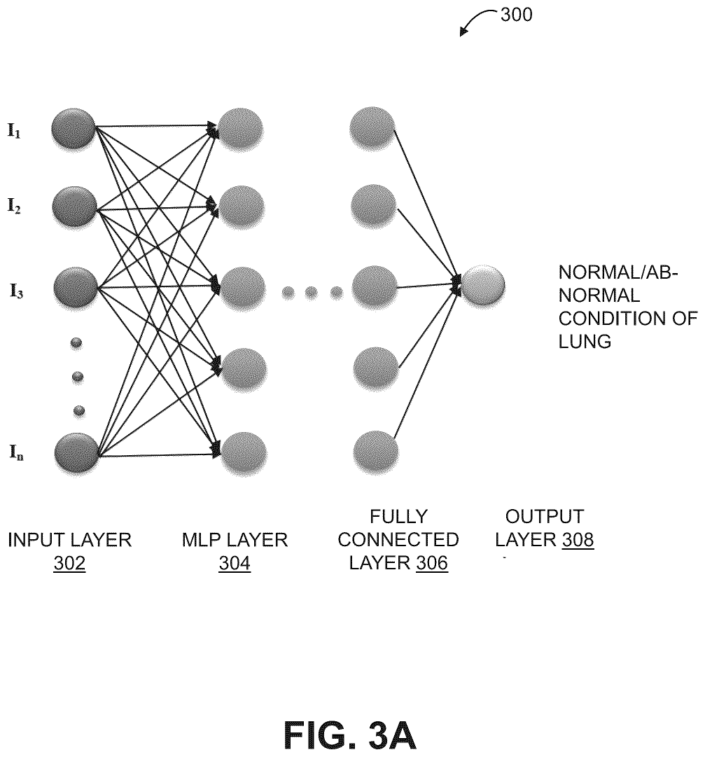

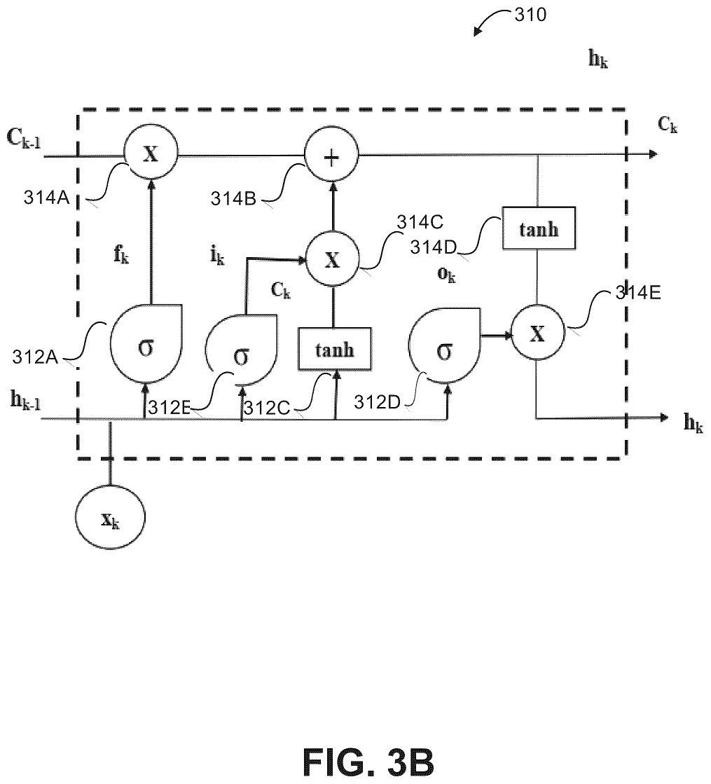

FIG. 3A illustrates an exemplary architectural representation of a multi-layer perceptron (MLP) neural network architecture 300 capable of processing unstructured dataset corresponding to a lung cancer, according to an example embodiment of the present disclosure. Consider, a scenario of diagnosing lung cancer through a feed-forward artificial neural network (ANN). The feed-forward ANN may include, for example, the MLP capable of learning long-term dependencies. The MLP networks are well-suited to classify, process, and predict based on the time-series data in the unstructured dataset corresponding to the lung cancer. The MLP may be a type of artificial neural network that may be composed of multiple layers of perceptron. Each perceptron in the MLP receives inputs from the previous layer, computes a weighted sum of the inputs, applies an activation function to the sum, and passes the result to the next layer.

The time-series data may include lags of unknown duration between important events in a time series. The vanishing gradient problem may be a common issue that can occur during the training of artificial neural networks. It occurs when the gradients of the loss function with respect to the parameters of the network become very small as they propagate through the layers of the network during back propagation. During back propagation, the gradients are multiplied by the weight matrices of each layer as they are propagated back through the network. If the weights are small, this multiplication can cause the gradients to become exponentially smaller as they are propagated back through the network. As a result, the parameters of the earlier layers in the network receive very small updates during training, which can lead to slower convergence and poor performance.

For example, the unstructured datasets may include a plurality of attributes (e.g., one thousand attributes) and for example four classes. The attributes in the unstructured dataset represent various genetic, molecular, and clinical features of the samples. These attributes may include gene expression levels, deoxyribonucleic acid (DNA) mutations, patient demographics, clinical outcomes, and the like. The four classes are adenocarcinomas (AD), squamous cell carcinomas (SQ), carcinoids (COID), and normal lung (NL). The AD and the SQ cell carcinomas are two of the most common types of lung cancer, while carcinoids are rare tumors that develop from neuroendocrine cells. The normal lung class represents healthy lung tissue. The dataset can be used for machine learning and data mining algorithms to develop models that accurately classify lung cancer samples based on their molecular and clinical characteristics. The models can potentially help clinicians diagnose and treat lung cancer more effectively, by identifying which patients are most likely to respond to specific treatments. Carcinoids are rare tumors that develop from neuroendocrine cells, and normal lung represents healthy lung tissue.

In addition, the MLP network may be trained using a gradient descent back. Traditional training techniques, on the other hand, have several disadvantages, such as sluggish convergence and the inability to discover the global minimum of the error function because gradient descent might become trapped in local minima. As a result, nature-inspired meta-heuristic algorithms are used to solve difficult problems in a derivative-free manner. The nature-inspired meta-heuristic algorithms are a class of optimization algorithms that are based on the principles of natural selection and evolution. These algorithms are designed to solve complex optimization problems by mimicking the behavior of biological systems such as genetic algorithms (GA), particle swarm optimization, ant colony optimization, artificial bee colony optimization, and many more. Considering all dependent variables into account, a supervised machine learning (ML) approaches enable for more accurate categorization and reduce physician misdiagnosis, which is estimated to occur about 20% of the time. Physician misdiagnosis occurs when a doctor or other healthcare professional fails to correctly identify a patient's medical condition. Misdiagnosis can occur for a variety of reasons, including incomplete or inaccurate medical histories, physical exams, and diagnostic tests.

The MLP may include arbitrary connections between neurons, usually fully connected between adjacent layers. The nodes of the network receive input from a current data point x (t) as well as the hidden state values of a hidden layer in a previous state h (t−1). Hence, inputs at a time ‘t’ have an impact on the outputs of the network to come in the future by the recurrent connections. A standard MLP with an input vector v=(v₁, . . . , v_{T}) calculates a hidden vector h=(h₁, . . . , h_{T}) and an output vector y=(y₁, . . . , y_{T}) by iterating equations 1 and 2 below, over t=1, . . . , T:

$$\\ \begin{matrix}h ( t ) = Q ( {W}_{( hx )} {x}^{{}_{} ( t )} + {W}_{( hh )} {h}^{{}_{} ( t - 1 )} + {b}_{h} ) & ( 1 )\end{matrix}$$

$$\\ \begin{matrix}{y}^{{}_{} ( t )} = σ ( {W}_{( yh )} {h}^{{}_{} ( t )} + {b}_{y} ) & ( 2 )\end{matrix}$$

In the above equations 1 and 2, the variable b_{y} and bₕ may be vectors of biases, and the variables W_{(h,x)}, W₍ₕₕ₎ and W_{(yh)} may be weights matrices of the input-hidden layer, hidden-output layer, and recurrent connections respectively. The variable ‘Q’ may be an activation function. The standard neural networks are trained across multiple time steps using the algorithm called backpropagation through time (BPTT). The BPTT may be an algorithm used for training MLPs) in which the gradients are calculated in reverse order over a sequence of inputs associated with the unstructured dataset corresponding to the lung cancer. In other words, it is a variation of the backpropagation algorithm that is designed to handle sequences of data, such as time series data. The BPTT works by unfolding the recurrent neural network over time, creating a feedforward neural network with a series of interconnected layers. The weights of the network are then updated using the backpropagation algorithm, which calculates the gradients of the loss function with respect to the weights at each timestep in the sequence. These gradients are then accumulated over the entire sequence and used to update the weights of the network.

The MLP neural network architecture 300 may include an input layer 302, a MLP layer 304, a fully connected layer 306, and an output layer 308, for processing unstructured dataset corresponding to a lung cancer. The output from the MLP neural network architecture 300 may be a normal or abnormal condition of the lung, based on the input of unstructured dataset corresponding to the lung cancer.

FIG. 3B illustrates a flow diagram representation of a long short-term memory (LSTM) cell 310 capable of processing unstructured dataset corresponding to a lung cancer, according to an example embodiment of the present disclosure.

In comparison to normal RNNs, the LSTM network model such as the LSTM cell 310 may acquire long-range relationships in actual applications. As a result, the LSTM cell 310 may be used in the majority of cutting-edge applications. The LSTM cell 310 may be made up of memory blocks in general. Memory cells and gates make up a memory block. Memory cells use self-connections to recall the network's temporal state, while gates govern the flow of information. Each memory block has an input gate to control the flow of input activations into the memory cell, an output gate to control the flow of cell activations out into the rest of the network, and a forget gate to control the flow of cell activations out of the network. The LSTM cell 310 may be held by the memory cell known as a ‘cell state’ that maintains its state over time. The cell state is the horizontal line that runs through the top of FIG. 3B. It can be visualized as a conveyor belt through which information just flows, unchanged. Information such as the unstructured data of the lung cancer can be added to or removed from the cell state in LSTM and is regulated by gates 314A and 314B. The gates 314A and 314B may optionally let the information flow in and out of the cell 310. Cell 310 includes a pointwise multiplication operation (X) 314C and 314E and a sigmoid neural net layer (σ) 312A, 312E, and 312D that assist the mechanism. The sigmoid layer (σ) gives out numbers between zero and one, where zero means ‘nothing should be let through,’ and one means ‘everything should be let through’. In the LSTM cell 310, a hyperbolic tangent function (tanh) 312C and 314D may be an activation function in several components of the network, which may be used to determine candidate cell state (internal state) values (Cₖ₋₁) and update the hidden state (hₖ₋₁). The ‘tanh’ function is used for the input gate, forget gate, and output gate. The input gate determines how much new information associated with the time-series data of the lung cancer is added to the cell state. The ‘tanh’ function is applied to the candidate input and the output of the input gate to create a new vector of values that can be added to the cell state. The forget gate determines how much information of the time-series data of the lung cancer from the previous cell state should be retained. The tanh function is applied to the output of the forget gate to create a new vector of values that can be multiplied with the previous cell state to determine which values should be forgotten. The output gate determines how much of the current cell state should be used to generate the output as the diagnosis of the lung cancer, of the LSTM. The tanh function is applied to the current cell state to produce a new vector of values that can be multiplied with the output gate to produce the final output corresponding to the diagnosis of the lung cancer.

For example, x (i) may be input vector to a unit of LSTM, and f (i) may be an activation vector of forget gate, f (j) means activation vector of input gate, o (i) means activation vector of output gate. The example of current and former step can be utilized in value of hidden layer's output vector ‘h’ and in internal state vector ‘s’. The network of LSTM understands when to allow their cells' internal states activation and when to allow their outputs activation. With their learning capability, all the gates in this gating mechanism can be taken as distinct components of LSTM cell 310. This implies that cells must adapt in process of training to maintain proper flow of information across the network as separate units. Hence, the internal state of cell can be unaffected while the gates are closed. To accomplish this, the function of hard sigmoid a was utilized and output is 0 and 1. Consequently, the gates are either fully closed or fully opened. Constant error carousel allows gradient for propagating back by several steps regarding backward pass.

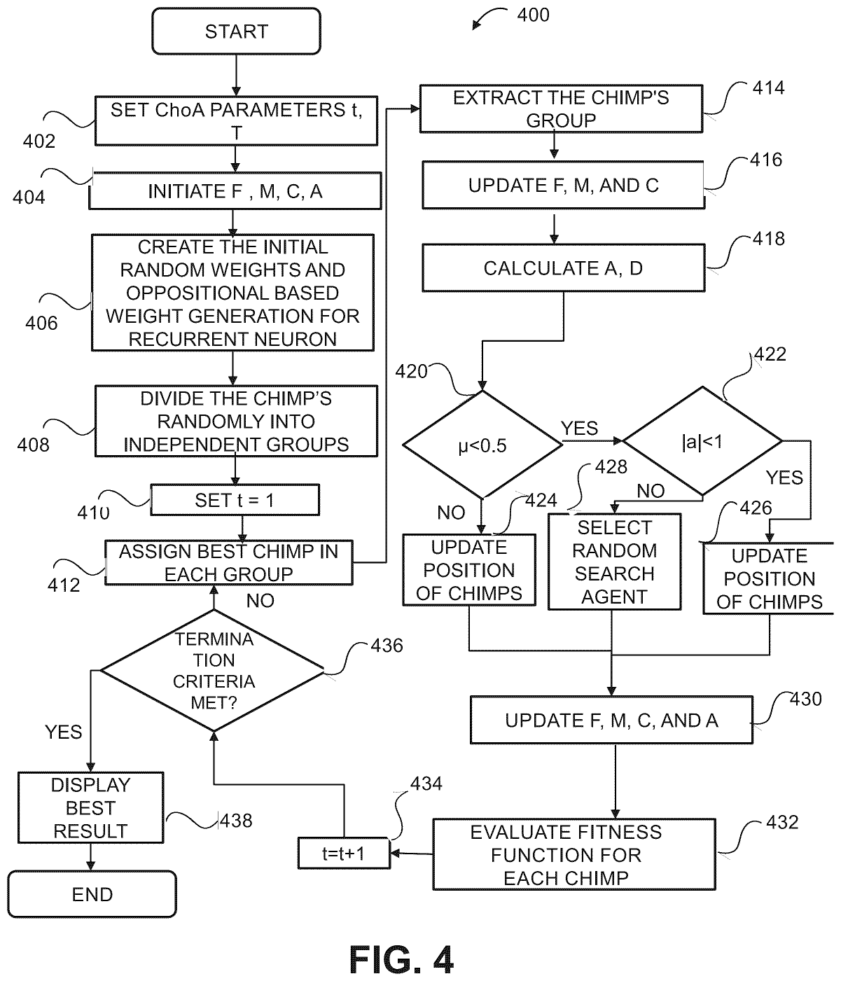

FIG. 4 illustrates a flow diagram representation of an oppositional-based chimp optimization method 400, according to an example embodiment of the present disclosure.

At step 402, the method 400 may include setting, by the system 102, chimp optimization Algorithm (ChoA) parameters t, T. The variable ‘t’ may be a time interval parameter for each iteration and variable ‘T’ may be a total time parameter for the chimp optimization model. At step 404, the method 400 may include initiating, by the system 102, the variables f, m, c, and a. The variable ‘f’ may be a fitness value of a candidate solution, variable ‘m’ may be a mean or an average fitness value of the population of candidate solutions, variable ‘c’ may be an exploration probability, and variable ‘a’ may be an acceleration coefficient, for the chimp optimization model, based on the set time interval parameter ‘t’ and the total time parameter ‘T’.

At step 406, the method 400 may include creating, by the system 102, initial random weights and generating oppositional-based weights for recurrent neurons of the RNN. At step 408, the method 400 may include dividing, by the system 102, the chimp's randomly into independent groups. At step 410, the method 400 may include setting, by the system 102, the variable ‘t’ (i.e., time interval parameter) to 1 for each iteration. At step 412, the method 400 may include assigning, by the system 102, the best chimps (best candidate solution) from each group of independent groups. At step 414, the method 400 may include extracting, by the system 102, the chimp's group. At step 416, the method 400 may include updating, by the system 102, variables f, m, and c using a group strategy. At step 418, the method 400 may include calculating, by the system 102, calculating variables ‘a’ and ‘d’, using f, m, and c. The variable ‘a’ and ‘d’, using f, m, and c may be calculated using equations 8 and 6 shown below sections.

At steps 420 and 422, the method 400 may include determining, by the system 102, if a mutation rate (p), and if a value of an inequality parameter (|a|) is greater than a pre-defined value (e.g., 0.5, 1, respectively) or lesser than a pre-defined value (e.g., 0.5, 1, respectively). The inequality parameter corresponds to a divergence of the candidate solutions, or a convergence toward the prey based on the value. When the mutation rate (p) is greater than a pre-defined value (e.g., 0.5), then the method 400 may include determining, by the system 102, if the inequality parameter (|a|) is lesser than the pre-defined value (e.g., 1).

At steps 424, and 426, the method 400 may include updating, by the system 102, a position of the chimps, when the mutation rate parameter is greater than the pre-defined value (0.5) and the value of the inequality parameter is lesser than a pre-defined value (1). The position of the chimps, when the mutation rate parameter is greater than the pre-defined value (0.5), is calculated using equation 12 shown in the below sections. The position of the chimps, when the value of the inequality parameter is lesser than a pre-defined value (1), is calculated using equation 7 shown in below equation 7. At step 428, the method 400 may include selecting, by the system 102, a random search agent, when the inequality parameter is greater than the pre-defined value (1).

At step 430, the method 400 may include updating, by the system 102, the fitness value of a candidate solution (f), the mean or average fitness value of the population of candidate solutions (m), the exploration probability (c), and the acceleration coefficient (a), based on the updated position of the chimps. At step 432, the method 400 may include evaluating, by the system 102, the fitness function for each chimp. At step 434, the method 400 may include incrementing, variable ‘t’ (i.e., time interval parameter). At step 436, the method 400 includes determining, by the system 102, if the termination criteria are met for the candidate solution based on the evaluated fitness function. The termination criteria correspond to at least one of a maximum number of iterations and a desired level of fitness value. At step 438, the method 400 includes displaying, by the system 102, on the user interface of the user device 106, the optimal chimp, when the termination criteria are met, and assigning, at step 412, the optimal chimp in each group, when the termination criteria are not met.

### Exemplary Scenario:

Consider, a chimp optimization algorithm (ChOA) such as the chimp optimization model as one of the metaheuristic algorithms. Each group of chimps (candidate solutions) devises its strategy for discovering the search space. In terms of ability and intellect, the chimps in each group are not all same, however, chimps are performing obligations as colony members. Each person's skill set might be valuable in a certain context. In a chimp colony, there are four types of chimps entitled such as a driver, barrier, chaser, and attackers. Each type of chimp has different abilities; however, the different abilities may be necessary for a successful hunt. Chimps can change duties during the same hunt or keep the same duty during the entire process. In addition to humans, the social incentives have been proposed only for chimps. This social incentive (sexual motivation) causes the chimps to act chaotically in the final stage of the hunting process.

The ChoA may implement one or more mathematical models for an independent group, a driving group, a blocking group, a chasing group, and an attacking group of chimps. Further, each of the one or more mathematical models may be initialized. For example, the population of chimps is initialized, where each chimp is considered as a potential solution to each solution. Each chimp indicates the values of the problem variables. The problem variables are considered as an array. In the context of mathematical modeling and optimization, problem variables refer to the parameters or factors that can be adjusted or optimized to achieve the desired outcome or objective. The choice of problem variables depends on the specific problem being considered, and they can be continuous or discrete, deterministic or stochastic, and constrained or unconstrained. For example, in an engineering design problem, the problem variables may include the dimensions of the product, the material properties, and the manufacturing parameters. In a transportation routing problem, the problem variables may include the distance between locations, the travel time, and the capacity of vehicles. In a financial portfolio optimization problem, the problem variables may include the allocation of funds to different investment options, the expected return, and the risk level.

The problem variables may be expressed as a “Nvariable”-dimensional optimization problem, a chimp group is an array of 1×Nvariable representing the solution of the problem. The array may be defined as shown in equation 3 below:

$$\\ \begin{matrix}chimp group = [ {I}_{1} , {I}_{2} , {I}_{3} , … {I}_{{N}_{variable}} ] & ( 3 )\end{matrix}$$

The solution length may be considered as the number of hidden units multiply by a number of input attributes in addition to a number of input attributes. Along with randomly generated weight parameters, the research involves opposition-based solution generation. The opposition-based solution (OBS) may be a technique used in optimization problems that aim to improve the performance of conventional optimization algorithms by using the concept of opposition. The idea is to generate new candidate solutions by taking the opposite of the current best candidate solutions in the search space, and then evaluating them to see if they improve the optimization process. The foremost goal behind opposition-based solution generation is the simultaneous consideration of corresponding opposite estimates as a second set of candidate solutions to achieve a better approximation for the current candidate solution. Further, an opposite candidate solution increases the chance to be closer to the global optimum solution than a randomly chosen candidate solution. The opposite candidate solution is a concept used in optimization algorithms that involves generating a new solution by taking the opposite of a given candidate solution in the search space. The idea behind this technique is that the opposite solution may represent a better option than the original solution as it may be more distant from it and hence may explore a different region of the search space. The opposition-based solution may be denoted as ‘NO’ which may be mathematically expressed as ins equation 4 below:

$$\\ \begin{matrix}{N}_{w ( j , i )}^{{}_{} o} = {x}_{i} + {y}_{i} - {N}_{w ( j , i )} & ( 4 )\end{matrix}$$

In the above equation 4, the opposition-based weights parameter solution refers to randomly generated weights. Both randomly generated solution and the opposition-based solution generations are fed into fitness computation for process evaluation. The randomly generated candidate solution is a technique used in optimization algorithms to generate an initial set of candidate solutions in the search space. The idea behind this technique is to generate a diverse set of solutions that explore different regions of the search space, without any prior knowledge or assumptions about the problem. The process of generating a randomly generated solution involves randomly assigning values to the decision variables within the bounds of the problem constraints. The values can be generated using various random number generation techniques, such as uniform or normal distributions, depending on the nature of the problem and the optimization algorithm being used.

### Fitness Function:

Fitness computation may be a process used in optimization algorithms to evaluate the quality of candidate solutions in the search space. The fitness function maps each candidate solution to a scalar value that represents its quality or fitness, based on the objective function and any constraints of the optimization problem. The fitness function is problem-specific and depends on the nature of the optimization problem and the objectives of the optimization algorithm. In general, the fitness function should be designed to accurately reflect the performance of the candidate solutions and guide the search process toward better solutions.

To start the optimization algorithm, a candidate widow matrix of size Npop×Nvar is generated with an initial random and opposition-based generated weight-parameters population of chimps. The Npop×Nvar refers to the size of the population in an optimization algorithm multiplied by the number of decision variables in the optimization problem. The system 102 selects the pairs of parents randomly to perform the procreating step by mating, in which the male black widow is eaten by the female during or after that. To evaluate the performance following mathematical function may be used as shown in equation 5 below:

$$\\ \begin{matrix}Accuracy = \frac{{T}_{P} + {T}_{N}}{{T}_{P} + {T}_{N} + {F}_{P} + {F}_{N}} & ( 5 )\end{matrix}$$

### Driving and Chasing the Prey:

Driving and chasing the prey may be a nature-inspired optimization algorithm based on the interaction between chimps and prey in ecosystems. The prey may be hunted during the exploration and exploitation phases. To mathematically model driving and chasing the prey, equations (6) and (7) are proposed as shown below:

$$\\ \begin{matrix}d = \vert c \cdot {x}_{prey} ( t ) - m \cdot {x}_{chimp} ( t ) \vert & ( 6 )\end{matrix}$$

$$\\ \begin{matrix}{x}_{chimp} ( t + 1 ) = {x}_{prey} ( t ) - a \cdot d & ( 7 )\end{matrix}$$

In the above equations 6 and 7, the variable ‘t’ may refer to the number of the current iteration. The variables a, m, and c are the coefficient vectors, the variable ‘Xprey’ may refer to the vector of prey position and the variable ‘Xchimp’ may be the position vector of a chimp. The vectors such as the a, m, and c vectors are calculated using below equations 8 to 10, respectively.

$$\\ \begin{matrix}a = 2 \cdot f \cdot {r}_{1} - f & ( 8 )\end{matrix}$$

$$\\ \begin{matrix}c = 2 \cdot {r}_{2} & ( 9 )\end{matrix}$$

$$\\ \begin{matrix}m = chaotic\_value & ( 10 )\end{matrix}$$

In which, the variable ‘f’ may be reduced non-linearly for example from 2.5 to 0 through the iteration process (in both the exploitation and the exploration phase). Where the variables ‘r1’ and ‘r2’ are the random vectors in the range of [0,1]. Finally, the variable ‘m’ is a chaotic vector calculated based on various chaotic maps so that this vector represents the effect of a sexual motivation of chimps in the hunting process. The concept can be generalized to n-dimensional search space. As mentioned in the previous section, the chimps also attack the prey with the chaotic strategy.

### Attacking Method (Exploitation Phase):

The chimps are capable of exploring the prey's location (by driving, blocking, and chasing) and then encircling the chimps. The hunting process may usually be conducted by attacker chimps. The driver, barrier, and chaser chimps are occasionally participating in the hunting process. Unfortunately, in an abstract search space, there is no information about the optimum location (prey). To mathematically simulate the behavior of the chimps, it may be assumed that the first attacker (best candidate solution available), driver, barrier, and chaser are better informed about the location of potential prey. For example, four of the best candidate solutions which are to be obtained may be stored and other chimps are forced to update the positions according to the best chimps' locations. This relationship is expressed using equations 11 to 13 shown below:

$$\\ \begin{matrix}{d}_{Attacker} = \vert {c}_{1} {x}_{Attacker} - {m}_{1} x \vert , {d}_{Barrier} = \vert {c}_{2} {x}_{Barrier} - {m}_{2} x \vert , & ( 11 )\end{matrix}$$

$$\\ {d}_{Chaser} = \vert {c}_{3} {x}_{chaser} - {m}_{3} x \vert , {d}_{Driver} = \vert {c}_{4} {x}_{Driver} - {m}_{4} x \vert .$$

$$\\ \begin{matrix}{x}_{1} = {x}_{Attacker} - {a}_{1} ( {d}_{Attacker} ) , {x}_{2} = {x}_{Barrier} - {a}_{2} ( {d}_{Barrier} ) , & ( 12 )\end{matrix}$$

$$\\ {x}_{3} = {x}_{Chaser} - {a}_{3} ( {d}_{Chaser} ) , {x}_{4} = {x}_{Driver} - {a}_{4} ( {d}_{Driver} ) .$$

$$\\ \begin{matrix}x ( t + 1 ) = \frac{{x}_{1} + {x}_{2} + {x}_{3} + {x}_{4}}{4} & ( 13 )\end{matrix}$$

In the above equations, the prey position may be estimated by the four best groups and other chimps randomly update the respective positions within the vicinity of chimps.

### Prey Attacking (Utilization):

In the final stage, the chimps may attack the prey and finish the hunt as soon as the prey stops moving. To mathematically model the attacking process, the value of the variable ‘f’ may be reduced. The variation range of the variable ‘a’ may also be reduced by the variable ‘f’. In other words, the variable ‘a’ may be a random variable in the interval of [−2f, 2f], while the value of ‘f’ reduces from 2.5 to 0 in the period of iterations. When the random values of a lie in the range of [−1, 1], the next position of a chimp can be in any location between its current position and the position of the prey.

The ChOA allows the chimps to update their positions according to the positions of attacker, barrier, chaser, and driver chimps and attack the prey. However, ChOAs may still be at the risk of trapping in local minima, so other operators are required to avoid this issue. Although, the proposed driving, blocking, and chasing mechanism somehow shows the exploration process, ChOA requires more operators to emphasize the exploration phase. Trapping in local minima is a common problem in optimization algorithms, including those inspired by nature such as the Chimp Optimization Algorithm (ChOA). Local minima are points in the search space where the objective function has a lower value than all of its neighboring points, but the overall value of the function is not optimal. When an optimization algorithm becomes trapped in a local minimum, it may converge prematurely and fail to find the globally optimal solution. In ChOA, trapping in local minima can occur when the population of candidate solutions becomes concentrated around a local minimum and fails to explore other regions of the search space. This can happen if the exploration and exploitation phases of the algorithm are not balanced or if the social learning aspect of the algorithm is not effective in sharing information about the global search space.

### Searching for Prey (Exploration Phase):

The exploration process among the chimps is mainly performed considering the location of the attacker, barrier, chaser, and driver chimps. They diverge to seek the prey and aggregate to attack prey. To mathematically model the divergence behavior, the ‘a’ vector with a random value greater than 1 or smaller than ‘−1’ may be used, so that the search agents are forced to diverge and get distant from prey. This procedure shows the exploration process and allows the ChOA to search globally. In the ChOA algorithm, an inequality ‘|a|>1’ forces the chimps to scatter in the environment to find better prey. The inequality |a|>1 may be used as a condition to trigger the “scattering” step, which allows the algorithm to explore the search space more widely and potentially find better candidate solutions. When a solution is evaluated and it satisfies the condition |a|>1, the algorithm will randomly scatter some of the chimps in the population to explore other areas of the search space. This helps to prevent the algorithm from getting stuck in local optima and encourages it to search globally for better candidate solutions.

Another ChOA component that affects the exploration phase is the value of the variable ‘c’. In ChOA, the c vector is a randomly generated vector that is used to control the balance between exploration and exploitation during the optimization process. It is not related to the position of the chimpanzee or any other aspect of the problem being optimized. The c vector is used to generate a set of weights that are applied to the candidate solutions in the population. These weights are used to determine the probability that each candidate solution is selected for exploration or exploitation in each iteration of the algorithm. The equation 9, the ‘c’ vector elements are random variables in the interval of [0, 2]. The ChOA component provides random weights for prey to reinforce (c>1) or lessen (c<1) the effect of prey location in the determination of the distance in equation 10. The ChOA component also helps ChOA to enhance its stochastic behavior along the optimization process and reduce the chance of trapping in local minima. The vector ‘c’ may always need to generate the random values and execute the exploration process not only in the initial iterations, but also in the final iterations. The factor may be very useful for avoiding local minima, especially in the final iterations. The vector ‘c’ may also be considered as the influence of the obstacles which prevent chimps from approaching the prey in nature. The natural obstacles in the path of chimps prevent the chimps from approaching the prey with proper speed. This is the precise expression of the ‘c’ vector effect. Depending on the chimp's position, the ‘c’ vector can assign a random weight to prey to make the hunt harder or easier.

### Social Incentive (Sexual Motivation):

Acquiring meet and subsequent social motivation (sex and grooming) in the final stage cause the chimps to release respective hunting responsibilities. Hence, the chimps try to obtain meat forcefully and chaotically. The chaotic behavior in the final stage helps chimps to further alleviate two problems of entrapment in local optima and a slow convergence rate in solving high-dimensional problems.

### Chaotic Maps:

The chaotic maps are mathematical functions that exhibit chaotic behavior, meaning that small changes in the initial conditions of the function can lead to dramatically different outputs. Chaotic maps are widely used in various fields, including physics, engineering, biology, and cryptography. Chaotic maps are often characterized by their sensitivity to initial conditions, their unpredictability, and their ability to generate complex and random patterns. Examples of chaotic maps include the logistic map, the Henon map, the Lorenz attractor, and the Rössler attractor. One of the main applications of chaotic maps is in the generation of pseudo-random numbers.

The chaotic maps improve the performance of the ChOA. For example, six chaotic maps may have been used, which are deterministic processes and also include random behavior. Consider, for example, value 0.7 as a primary point of all the chaotic maps. To model the simultaneous behavior, assume that there is a probability of 50% to choose between either the normal updating position mechanism or the chaotic model to update the position of chimps during optimization. The normal updating position mechanism is a common approach used in optimization algorithms, including the Chimp Optimization Algorithm (ChOA), to update the position of candidate solutions during the search process. The mathematical model may be expressed using equation 14 below:

$$\\ \begin{matrix}{x}_{chimp} ( t + 1 ) = \{ \begin{matrix}{x}_{prey} ( t ) - a \cdot d if μ < 0.5 \\ Chaotic\_value if μ \geq 0.5\end{matrix} & ( 14 )\end{matrix}$$

In the above equation 14, the variable ‘μ’ may be a random number in [0, 1].

The searching process in the ChOA may begin with generating a stochastic population of chimps (such as the candidate solutions). Further, all chimps may be randomly divided into four predefined independent groups such as, but not limited to, attacker, barrier, chaser, driver, and the like. Each chimp updates respective ‘f’ coefficients using the respective group strategy. During the iteration period, attacker, barrier, chaser, and driver chimps may estimate the possible prey locations. Each candidate solution updates the respective distance from the prey. Adaptive tuning of the vectors ‘c’ and ‘m’ may cause local optima avoidance and a faster convergence curve, simultaneously. The value of ‘f’ may be reduced from 2.5 to zero, to enhance the process of exploitation and attacking the prey. The inequality |a|>1 may result in divergence of the candidate solutions, otherwise, the candidate solutions eventually converge toward the prey.

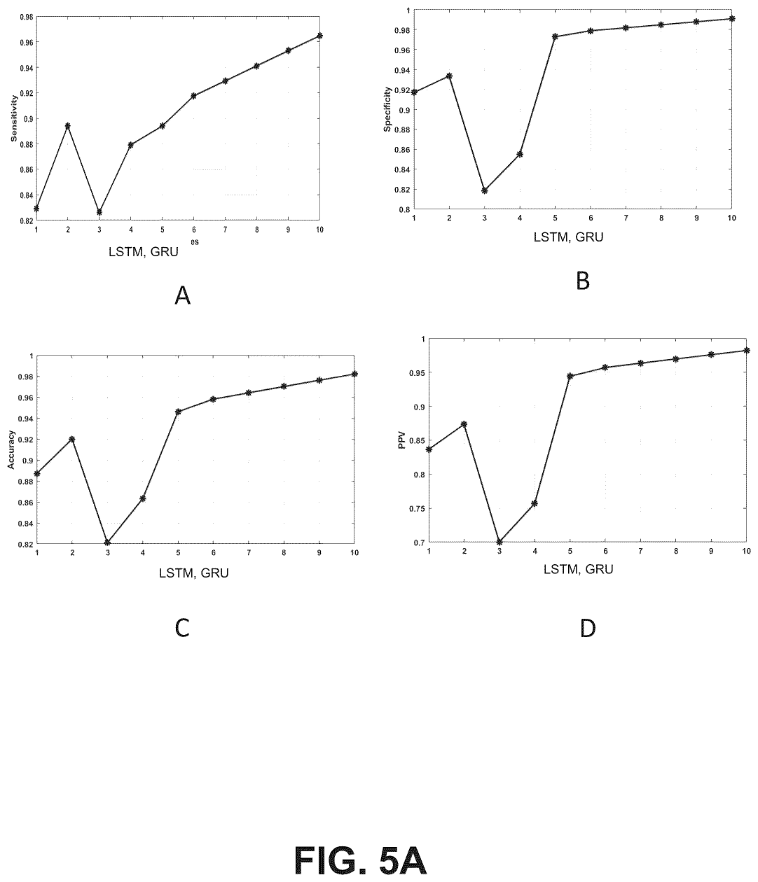

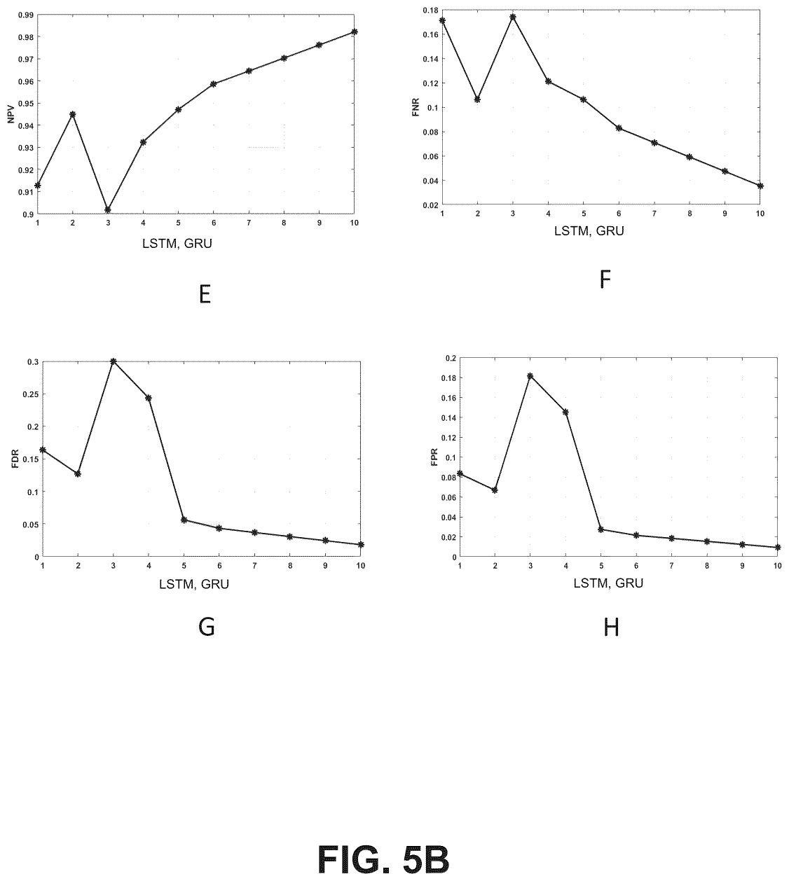

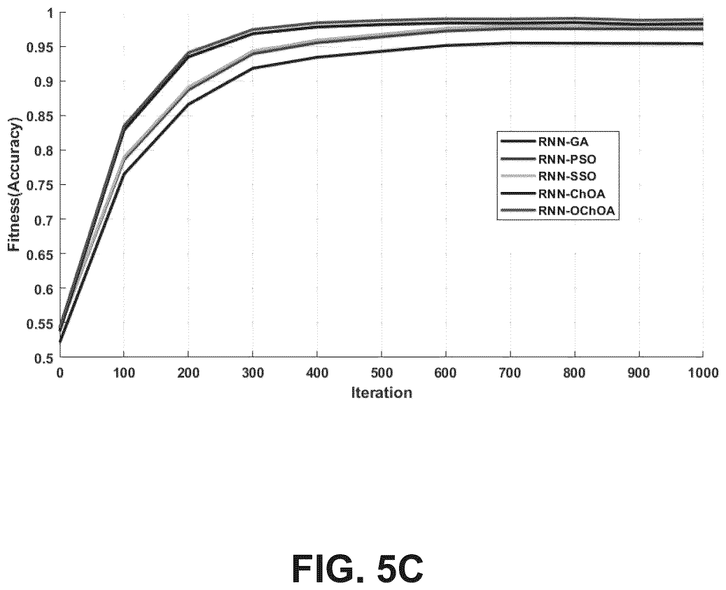

FIGS. 5A and 5B illustrate graphical representations of a performance of contest techniques for different measures, according to an example embodiment of the present disclosure.

For example, diagnosing lung conditions of the individual, using the RNN with optimal weights from optimized ChOA may achieve better performance over other comparative techniques. The performance of the involved techniques evaluates through eight different standard measures based on the results shown in the graphs FIGS. 5A and 5B that the proposed approach has better performance in all measures. The context techniques involved may be shown in table 1 below.

TABLE 1

| S. No | Techniques |
|---|---|
| 1 | Radial Basis Neural Network (RBNN) |
| 2 | Feed Forward Back Propagation Neural |
| | Network (FBNN) |
| 3 | k-Nearest Neighbours (KNN) |
| 4 | Random Forest (RF) |
| 5 | Recurrent Neural Network (RNN) |
| 6 | RNN-GA |
| 7 | RNN-PSO |
| 8 | RNN-SSO |
| 9 | RNN-ChOA |
| 10 | RNN-OChOA |

Further, the considered performance measures of techniques such as a long short-term memory networks (LSTM) and a gated recurrent unit (GRU) are, but not limited to, a sensitivity measure (graph A), a specificity measure (graph B), an accuracy measure (graph C), a positive predictive value (PPV) measure (graph D), a negative predictive value (NPV) measure (graph E), a false negative rate (FNR) measure (graph F), a false discovery rate (FDR) measure (graph G), a false positive rate (FPR) measure (graph H), and the like, as shown in Table 2 below.

TABLE 2

| Measures | Values |
|---|---|
| Sensitivity | 0.964602 |
| Specificity | 0.990923 |
| Accuracy | 0.982 |
| PPV | 0.981982 |
| NPV | 0.982009 |
| FPR | 0.009077 |
| FNR | 0.035398 |
| FDR | 0.018018 |

Involving oppositional-based solution generation along with random solution generation elevates the traditional ChOA performance. Opposition-based learning as a scheme for machine learning intelligence that estimates and counter-estimates, weights and opposite weights, and actions versus counter-actions are the foundation of this approach. The proposed techniques using the system 102 may show that OChOA involved in configuring optimal weights achieves the accuracy of, for example, 98.2% accuracy; which may be for example 0.6% better than traditional ChOA, integrate into finding optimal weights, 1.2% greater than SSO, 1.8% higher than PSO, 2.4% better than GA. While comparing the proposed technique performance with traditional classification techniques it is 3.6% better accuracy than RNN, it is 11.9% greater accuracy over RF, 16.1% higher accuracy than KNN, 6.2% better accuracy over FBNN, and 9.5% greater accuracy over RBNN. In the case of considering other measures, the proposed OChOA associates in finding optimal weights for RNN achieve superior performance in all employed measures.

FIG. 5C illustrates a graphical representation of a converging performance of optimization techniques involved in finding optimal weights, according to an example embodiment of the present disclosure. The graph is shown in FIG. 5C provides converging performance of involved techniques during training time. This investigation process from, for example, 0 to 1000 iterations concerning fitness. From the graph, it may be evident from the results that the proposed optimized OChOA is associated with identifying optimal weights for RNN converge at 500th iterations, which may be close to the traditional ChOA technique. The early converging between the 400th to 500th iteration may be possible because of integrating the opposition strategy along with the initially generated random-based solution in traditional ChOA.

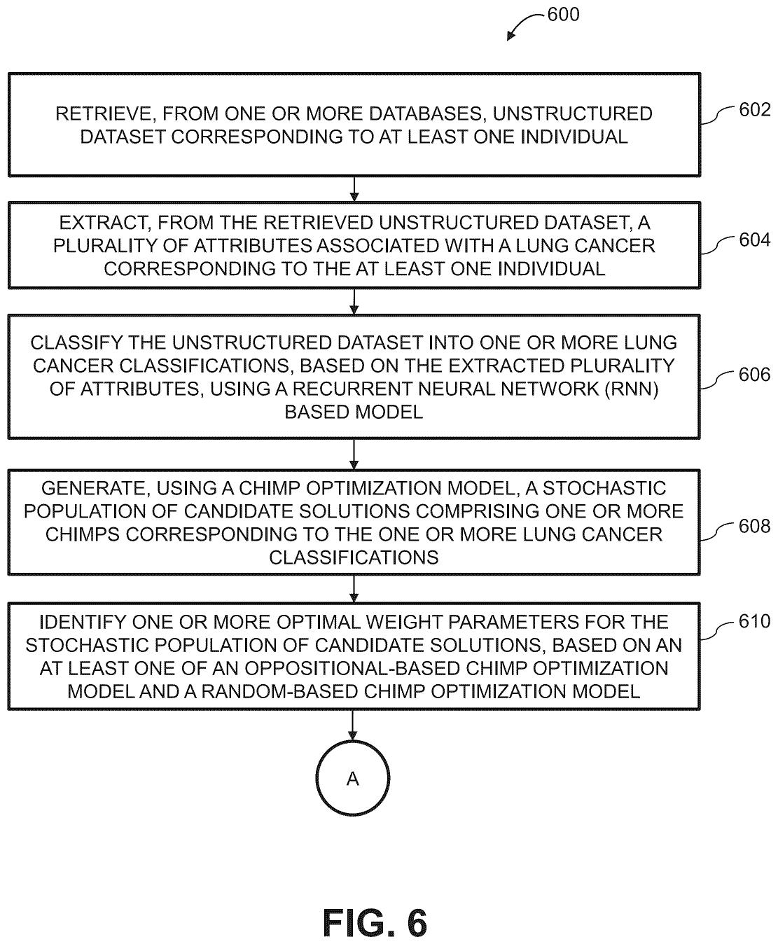

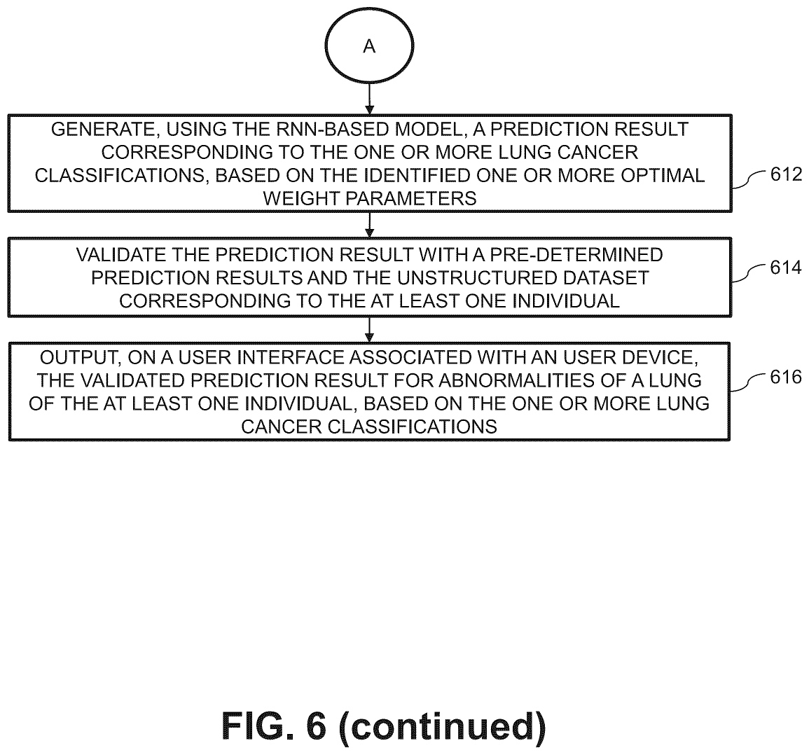

FIG. 6 illustrates a flow chart depicting a method 600 for processing unstructured dataset corresponding to a lung cancer, according to an example embodiment of the present disclosure.

At block 602, the method 600 may include retrieving, by one or more hardware processors 110, from one or more databases, unstructured dataset corresponding to at least one individual. The unstructured dataset comprises at least one of single time-point data and time-series data. At block 604, the method 600 may include extracting, by the one or more hardware processors 110, from the retrieved unstructured dataset, a plurality of attributes associated with a lung cancer corresponding to the at least one individual.

At block 606, the method 600 may include classifying, by the one or more hardware processors 110, the unstructured dataset into one or more lung cancer classifications, based on the extracted plurality of attributes, using a Recurrent Neural Network (RNN) based model. At block 608, the method 600 may include generating, by the one or more hardware processors 110, using a chimp optimization model, a stochastic population of candidate solutions comprising one or more chimps corresponding to the one or more lung cancer classifications.

At block 610, the method 600 may include identifying, by the one or more hardware processors 110, one or more optimal weight parameters for the stochastic population of candidate solutions, based on an at least one of an oppositional-based chimp optimization model and a random-based chimp optimization model. The one or more optimal weight parameters are identified for a convergence of the RNN-based model. At block 612, the method 600 may include generating, by the one or more hardware processors 110, using the RNN-based model, a prediction result corresponding to the one or more lung cancer classifications, based on the identified one or more optimal weight parameters.

At block 614, the method 600 may include validating, by the one or more hardware processors 110, the prediction result with a pre-determined prediction results and the unstructured dataset corresponding to the at least one individual. At block 616, the method 600 may include outputting, by the one or more hardware processors 110, on a user interface associated with the user device 106, the validated prediction result for abnormalities of a lung of the at least one individual, based on the one or more lung cancer classifications.

The method 600 may be implemented in any suitable hardware, software, firmware, or combination thereof. The order in which the method 600 is described is not intended to be construed as a limitation, and any number of the described method blocks may be combined or otherwise performed in any order to implement the method 600 or an alternate method. Additionally, individual blocks may be deleted from the method 600 without departing from the spirit and scope of the present disclosure described herein. Furthermore, the method 600 may be implemented in any suitable hardware, software, firmware, or a combination thereof, that exists in the related art or that is later developed. The method 600 describes, without limitation, the implementation of the system 102. A person of skill in the art will understand that method 600 may be modified appropriately for implementation in various manners without departing from the scope and spirit of the disclosure.

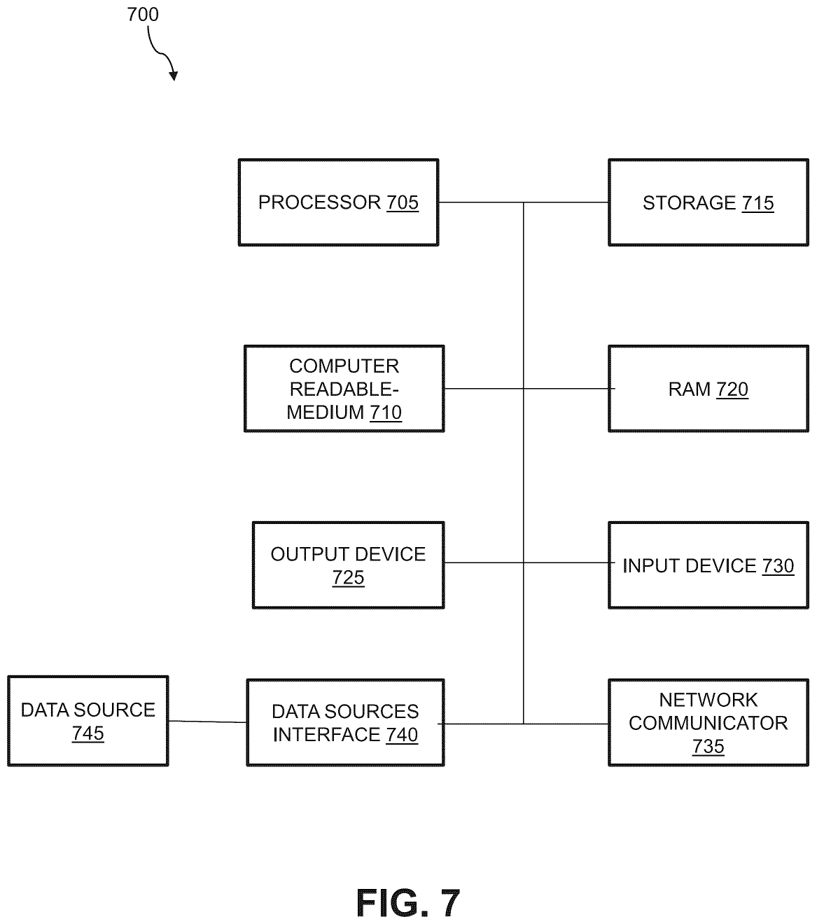

FIG. 7 illustrates an exemplary block diagram representation of a hardware platform 700 for implementation of the disclosed system 102, according to an example embodiment of the present disclosure. For the sake of brevity, the construction, and operational features of the system 102 which are explained in detail above are not explained in detail herein. Particularly, computing machines such as but not limited to internal/external server clusters, quantum computers, desktops, laptops, smartphones, tablets, and wearables which may be used to execute the system 102 or may include the structure of the hardware platform 700. As illustrated, the hardware platform 700 may include additional components not shown, and some of the components described may be removed and/or modified. For example, a computer system with multiple GPUs may be located on external-cloud platforms including Amazon Web Services, or internal corporate cloud computing clusters, or organizational computing resources.

The hardware platform 700 may be a computer system such as the system 106 that may be used with the embodiments described herein. The computer system may represent a computational platform that includes components that may be in a server or another computer system. The computer system may execute, by the processor 705 (e.g., single or multiple processors) or other hardware processing circuits, the methods, functions, and other processes described herein. These methods, functions, and other processes may be embodied as machine-readable instructions stored on a computer-readable medium, which may be non-transitory, such as hardware storage devices (e.g., RAM (random access memory), ROM (read-only memory), EPROM (erasable, programmable ROM), EEPROM (electrically erasable, programmable ROM), hard drives, and flash memory). The computer system may include the processor 705 that executes software instructions or code stored on a non-transitory computer-readable storage medium 710 to perform methods of the present disclosure. The software code includes, for example, instructions to gather data and analyze the data. For example, the browser agent subsystem 114 includes the data retrieving subsystem 206, the attribute extracting subsystem 208, the classifying subsystem 210, the solution generating subsystem 212, the parameter identifying subsystem 214, the result generating subsystem 216, the validating subsystem 218, and the result outputting subsystem 220.

The instructions on the computer-readable storage medium 710 are read and stored the instructions in storage 715 or random-access memory (RAM). The storage 715 may provide a space for keeping static data where at least some instructions could be stored for later execution. The stored instructions may be further compiled to generate other representations of the instructions and dynamically stored in the RAM such as RAM 720. The processor 705 may read instructions from the RAM 720 and perform actions as instructed.

The computer system may further include the output device 725 to provide at least some of the results of the execution as output including, but not limited to, visual information to users, such as external agents. The output device 725 may include a display on computing devices and virtual reality glasses. For example, the display may be a mobile phone screen or a laptop screen. GUIs and/or text may be presented as an output on the display screen. The computer system may further include an input device 730 to provide a user or another device with mechanisms for entering data and/or otherwise interacting with the computer system. The input device 730 may include, for example, a keyboard, a keypad, a mouse, or a touchscreen. Each of these output devices 725 and input device 730 may be joined by one or more additional peripherals. For example, the output device 725 may be used to display the results such as bot responses by the executable chatbot.

A network communicator 735 may be provided to connect the computer system to a network and in turn to other devices connected to the network including other clients, servers, data stores, and interfaces, for example. A network communicator 735 may include, for example, a network adapter such as a LAN adapter or a wireless adapter. The computer system may include a data sources interface 740 to access the data source 745. The data source 745 may be an information resource. As an example, a database of exceptions and rules may be provided as the data source 745. Moreover, knowledge repositories and curated data may be other examples of the data source 745.

The written description describes the subject matter herein to enable any person skilled in the art to make and use the embodiments. The scope of the subject matter embodiments is defined by the claims and may include other modifications that occur to those skilled in the art. Such other modifications are intended to be within the scope of the claims if they have similar elements that do not differ from the literal language of the claims or if they include equivalent elements with insubstantial differences from the literal language of the claims.

The embodiments herein can comprise hardware and software elements. The embodiments that are implemented in software include but are not limited to, firmware, resident software, microcode, etc. The functions performed by various modules described herein may be implemented in other modules or combinations of other modules. For the purposes of this description, a computer-usable or computer-readable medium can be any apparatus that can comprise, store, communicate, propagate, or transport the program for use by or in connection with the instruction execution system, apparatus, or device.

A description of an embodiment with several components in communication with each other does not imply that all such components are required. On the contrary, a variety of optional components are described to illustrate the wide variety of possible embodiments of the invention. When a single device or article is described herein, it will be apparent that more than one device/article (whether or not they cooperate) may be used in place of a single device/article. Similarly, where more than one device or article is described herein (whether or not they cooperate), it will be apparent that a single device/article may be used in place of the more than one device or article, or a different number of devices/articles may be used instead of the shown number of devices or programs. The functionality and/or the features of a device may be alternatively embodied by one or more other devices which are not explicitly described as having such functionality/features. Thus, other embodiments of the invention need not include the device itself.

The illustrated steps are set out to explain the exemplary embodiments shown, and it should be anticipated that ongoing technological development will change the manner in which particular functions are performed. These examples are presented herein for purposes of illustration, and not limitation. Further, the boundaries of the functional building blocks have been arbitrarily defined herein for the convenience of the description. Alternative boundaries can be defined so long as the specified functions and relationships thereof are appropriately performed. Alternatives (including equivalents, extensions, variations, deviations, etc., of those described herein) will be apparent to persons skilled in the relevant art(s) based on the teachings contained herein. Such alternatives fall within the scope and spirit of the disclosed embodiments. Also, the words “comprising,” “having,” “containing,” and “including,” and other similar forms are intended to be equivalent in meaning and be open-ended in that an item or items following any one of these words is not meant to be an exhaustive listing of such item or items or meant to be limited to only the listed item or items. It must also be noted that as used herein and in the appended claims, the singular forms “a,” “an,” and “the” include plural references unless the context clearly dictates otherwise.

Finally, the language used in the specification has been principally selected for readability and instructional purposes, and it may not have been selected to delineate or circumscribe the inventive subject matter. It is therefore intended that the scope of the invention be limited not by this detailed description, but rather by any claims that issue on an application based here on. Accordingly, the embodiments of the present invention are intended to be illustrative, but not limited, of the scope of the invention, which is outlined in the following claims.

What is claimed is:

1. A computer-implemented system for processing unstructured dataset corresponding to a lung cancer, the computer-implemented system comprising:
one or more hardware processors; and
a memory coupled to the one or more hardware processors, wherein the memory comprises a plurality of subsystems in form of programmable instructions executable by the one or more hardware processors, wherein the plurality of subsystems comprises:
a data retrieving subsystem configured to retrieve, from one or more databases, unstructured dataset corresponding to at least one individual, wherein the unstructured dataset comprises at least one of single time-point data and time-series data;
an attribute extracting subsystem configured to extract, from the retrieved unstructured dataset, a plurality of attributes associated with a lung cancer corresponding to the at least one individual;
a classifying subsystem configured to classify the unstructured dataset into one or more lung cancer classifications, based on the extracted plurality of attributes, using a Recurrent Neural Network (RNN) based model;
a solution-generating subsystem configured to generate, using a chimp optimization model, a stochastic population of candidate solutions comprising one or more chimps corresponding to the one or more lung cancer classifications;
a parameter identifying subsystem configured to identify one or more optimal weight parameters for the stochastic population of candidate solutions, based on an at least one of an oppositional-based chimp optimization model and a random-based chimp optimization model, wherein the one or more optimal weight parameters are identified for a convergence of the RNN based model;
a result-generating subsystem configured to generate, using the RNN-based model, a prediction result corresponding to the one or more lung cancer classifications, based on the identified one or more optimal weight parameters;
a validating subsystem configured to validate the prediction result with a pre-determined prediction results and the unstructured dataset corresponding to the at least one individual; and
a result outputting subsystem configured to output, on a user interface associated with a user device, the validated prediction result for abnormalities of a lung of the at least one individual, based on the one or more lung cancer classifications.

2. The computer-implemented system of claim 1, wherein, for identifying one or more optimal weight parameters, the plurality of subsystems further comprises:
a parameter setting subsystem configured to set a time interval parameter for each iteration and a total time parameter for the chimp optimization model;
a value initiating subsystem configured to initiate a fitness value of a candidate solution (f), a mean or average fitness value of the population of candidate solutions (m), an exploration probability (c), and an acceleration coefficient (a), for the chimp optimization model, based on the set time interval parameter and the total time parameter;
a parameter-creating subsystem configured to create initial random weight parameters and generate oppositional weight parameters for the convergence of the RNN-based model;
a segregating subsystem configured to segregate randomly, the one or more chimps into one or more independent groups, wherein the independent groups comprise at least one of an attacker, a barrier, a chaser, and a driver;
a group assigning subsystem configured to assign in each independent group, an optimal chimp from the one or more chimps;
a group extracting subsystem configured to extract each independent group of the assigned optimal chimp;
a value updating subsystem configured to update the initiated fitness value of the candidate solution (f), the mean or average fitness value of the population of candidate solutions (m), and the exploration probability (c), of each of the one or more chimps, based on a group strategy of the extracted each independent group;
a calculating subsystem configured to calculate the acceleration coefficient (a), and a dimensionality (d) of a search space, based on the updated fitness value of the candidate solution (f), the mean or average fitness value of the population of candidate solutions (m), and the exploration probability (c);
a determining subsystem configured to determine, if a mutation rate and if a value of an inequality parameter is greater than a pre-defined value or lesser than a pre-defined value, wherein the inequality parameter corresponds to a divergence of the candidate solutions, or a convergence toward the prey based on the value;
a position updating subsystem configured to update a position of the chimps, when the mutation rate and the value of the inequality parameter are greater than the pre-defined value;
the position updating subsystem configured to update the position of each chimp, and select a random search agent, when the mutation rate and the value of the inequality parameter are lesser than the pre-defined value;
the value updating subsystem configured to update the fitness value of a candidate solution (f), the mean or average fitness value of the population of candidate solutions (m), the exploration probability (c), and the acceleration coefficient (a), based on the updated position of the chimps;
a function evaluating subsystem configured to evaluate the fitness function for each chimp;
a criteria-determining subsystem configured to determine if the termination criteria are met for the candidate solution based on the evaluated fitness function, wherein the termination criteria corresponds to at least one of a maximum number of iterations and a desired level of fitness value; and
a displaying subsystem configured to display on the user interface of the user device, the optimal chimp, when the termination criteria are met, and assign optimal chimp in each group, when the termination criteria are not met.

3. The computer-implemented system of claim 2, wherein the plurality of subsystems further comprises:
an attack-triggering subsystem configured to trigger the one or more chimps to attack one or more prey using a chaotic strategy comprising one or more chaotic maps, wherein the one or more prey corresponds to the candidate solutions;
a location estimating subsystem configured to estimate possible locations of the one or more prey by the one or more independent groups of chimps comprising the attacker, the barrier, the chaser, and the driver;
a distance updating subsystem configured to update the distance of each candidate solution from the one or more prey, based on the estimated possible locations of the one or more prey; and
a value decrementing subsystem configured to decrement a value of fitness value (f) of the candidate solution to increase an exploitation probability of attacking the one or more prey.

4. The computer-implemented system of claim 3, wherein the one or more prey is attacked during at least one of an exploration phase and an exploitation phase.

5. The computer-implemented system of claim 4, wherein the exploration phase corresponds to searching for the one or more prey by a driving operation, a blocking operation, and a chasing operation, and the exploitation phase corresponds to an attacking operation of the one or more prey.

6. The computer-implemented system of claim 3, wherein the one or more chaotic maps update the position of the one or more chimps.

7. The computer-implemented system of claim 1, wherein the plurality of subsystems further comprises:
a data extracting subsystem configured to extract the single time-point data and the time-series data from at least one of medical images, medical records, patient information, clinical laboratory test results, electronic health records, clinical notes, physician reports, and patient surveys, retrieved from the one or more databases.

8. The computer-implemented system of claim 1, wherein the plurality of subsystems further comprises:
a training subsystem to train the RNN-based model, using the chimp optimization model and a backpropagation through time model, to classify the one or more lung cancer classes based on the time series data from the unstructured dataset.

9. The computer-implemented system of claim 1, wherein the one or more lung cancer classifications comprise at least one of an adenocarcinomas (AD) class, a squamous cell carcinomas (SQ) class, a carcinoids (COID) class, and a normal lung (NL) class.

10. A computer-implemented method for processing unstructured dataset corresponding to a lung cancer, the computer-implemented method comprising:
retrieving, by one or more hardware processors, from one or more databases, unstructured dataset corresponding to at least one individual, wherein the unstructured dataset comprises at least one of single time-point data and time-series data;
extracting, by the one or more hardware processors, from the retrieved unstructured dataset, a plurality of attributes associated with a lung cancer corresponding to the at least one individual;
classifying, by the one or more hardware processors, the unstructured dataset into one or more lung cancer classifications, based on the extracted plurality of attributes, using a Recurrent Neural Network (RNN) based model;
generating, by the one or more hardware processors, using a chimp optimization model, a stochastic population of candidate solutions comprising one or more chimps corresponding to the one or more lung cancer classifications;
identifying, by the one or more hardware processors, one or more optimal weight parameters for the stochastic population of candidate solutions, based on an at least one of an oppositional-based chimp optimization model and a random-based chimp optimization model, wherein the one or more optimal weight parameters are identified for a convergence of the RNN based model;
generating, by the one or more hardware processors, using the RNN-based model, a prediction result corresponding to the one or more lung cancer classifications, based on the identified one or more optimal weight parameters;
validating, by the one or more hardware processors, the prediction result with a pre-determined prediction results and the unstructured dataset corresponding to the at least one individual; and
outputting, by the one or more hardware processors, on a user interface associated with a user device, the validated prediction result for abnormalities of a lung of the at least one individual, based on the one or more lung cancer classifications.

11. The computer-implemented method of claim 10, wherein, for identifying one or more optimal weight parameters, the method further comprises:
setting, by the one or more hardware processors, a time interval parameter for each iteration and a total time parameter for the chimp optimization model;
initiating, by the one or more hardware processors, a fitness value of a candidate solution (f), a mean or average fitness value of the population of candidate solutions (m), an exploration probability (c), and an acceleration coefficient (a), for the chimp optimization model, based on the set time interval parameter and the total time parameter,
creating, by the one or more hardware processors, initial random weight parameters and generating oppositional weight parameters for the convergence of the RNN-based model;
segregating randomly, by the one or more hardware processors, the one or more chimps into one or more independent groups, wherein the independent groups comprise at least one of an attacker, a barrier, a chaser, and a driver;
assigning, by the one or more hardware processors, in each independent group, an optimal chimp from the one or more chimps;
extracting, by the one or more hardware processors, each independent group of the assigned optimal chimp;
updating, by the one or more hardware processors, the initiated fitness value of the candidate solution (f), the mean or average fitness value of the population of candidate solutions (m), and the exploration probability (c), of each of the one or more chimps, based on a group strategy of the extracted each independent group;
calculating, by the one or more hardware processors, the acceleration coefficient (a), and a dimensionality (d) of a search space, based on the updated fitness value of the candidate solution (f), the mean or average fitness value of the population of candidate solutions (m), and the exploration probability (c);
determining, by the one or more hardware processors, if a mutation rate and if a value of an inequality parameter is greater than a pre-defined value or lesser than a pre-defined value, wherein the inequality parameter corresponds to a divergence of the candidate solutions, or a convergence toward the prey based on the value;
updating, by the one or more hardware processors, a position of the chimps, when the mutation rate and the value of the inequality parameter is greater than the pre-defined value;
updating, by the one or more hardware processors, the position of each chimp, and selecting a random search agent, when the mutation rate and the value of the inequality parameter are lesser than the pre-defined value;
updating, by the one or more hardware processors, the fitness value of a candidate solution (f), the mean or average fitness value of the population of candidate solutions (m), the exploration probability (c), and the acceleration coefficient (a), based on the updated position of the chimps;
evaluating, by the one or more hardware processors, the fitness function for each chimp;
determining, by the one or more hardware processors, if the termination criteria are met for the candidate solution based on the evaluated fitness function, wherein the termination criteria corresponds to at least one of a maximum number of iterations and a desired level of fitness value; and
displaying, by the one or more hardware processors, on the user interface of the user device, the optimal chimp, when the termination criteria are met, and assigning optimal chimp in each group, when the termination criteria are not met.

12. The computer-implemented method of claim 10, wherein the method further comprises:
trigger, by the one or more hardware processors, the one or more chimps to attack one or more prey using a chaotic strategy comprising one or more chaotic maps, wherein the one or more prey corresponds to the candidate solutions;
estimating, by the one or more hardware processors, possible locations of the one or more prey by the one or more independent groups of chimps comprising the attacker, the barrier, the chaser, and the driver;
updating, by the one or more hardware processors, the distance of each candidate solution from the one or more prey, based on the estimated possible locations of the one or more prey; and
decrementing, by the one or more hardware processors, a value of fitness value (f) of the candidate solution to increase an exploitation probability of attacking the one or more prey.

13. The computer-implemented method of claim 12, wherein the one or more prey is attacked during at least one of an exploration phase and an exploitation phase.

14. The computer-implemented method of claim 13, wherein the exploration phase corresponds to searching for the one or more prey by a driving operation, a blocking operation, and a chasing operation, and the exploitation phase corresponds to an attacking operation of the one or more prey.

15. The computer-implemented method of claim 12, wherein the one or more chaotic maps update the position of the one or more chimps.

16. The computer-implemented method of claim 10, wherein the method further comprises:
extracting, by the one or more hardware processors, the single time-point data and the time-series data from at least one of medical images, medical records, patient information, clinical laboratory test results, electronic health records, clinical notes, physician reports, and patient surveys, retrieved from the one or more databases.

17. The computer-implemented method of claim 10, wherein the method further comprises:
training, by the one or more hardware processors, the RNN-based model, using the chimp optimization model and a backpropagation through time model, to classify the one or more lung cancer classes based on the time series data from the unstructured dataset.

18. The computer-implemented method of claim 10, wherein the one or more lung cancer classifications comprise at least one of an adenocarcinomas (AD) class, a squamous cell carcinomas (SQ) class, a carcinoids (COID) class, and a normal lung (NL) class.

19. A non-transitory computer-readable storage medium having instructions stored therein that, when executed by one or more hardware processors, cause the one or more hardware processors to:
retrieve, from one or more databases, unstructured dataset corresponding to at least one individual, wherein the unstructured dataset comprises at least one of single time-point data and time-series data;
extract, from the retrieved unstructured dataset, a plurality of attributes associated with a lung cancer corresponding to the at least one individual;
classify the unstructured dataset into one or more lung cancer classifications, based on the extracted plurality of attributes, using a Recurrent Neural Network (RNN) based model;
generate, using a chimp optimization model, a stochastic population of candidate solutions comprising one or more chimps corresponding to the one or more lung cancer classifications;
identify one or more optimal weight parameters for the stochastic population of candidate solutions, based on an at least one of an oppositional-based chimp optimization model and a random-based chimp optimization model, wherein the one or more optimal weight parameters are identified for a convergence of the RNN based model;
generate, using the RNN-based model, a prediction result corresponding to the one or more lung cancer classifications, based on the identified one or more optimal weight parameters;
validate the prediction result with a pre-determined prediction results and the unstructured dataset corresponding to the at least one individual; and
output, on a user interface associated with a user device, the validated prediction result for abnormalities of a lung of the at least one individual, based on the one or more lung cancer classifications.

20. The non-transitory computer-readable storage medium of claim 19, wherein, for identifying one or more optimal weight parameters, the one or more processor is further configured to:
set a time interval parameter for each iteration and a total time parameter for the chimp optimization model;
initiate a fitness value of a candidate solution (f), a mean or average fitness value of the population of candidate solutions (m), an exploration probability (c), and an acceleration coefficient (a), for the chimp optimization model, based on the set time interval parameter and the total time parameter;
create initial random weight parameters and generate oppositional weight parameters for the convergence of the RNN-based model;
segregate randomly, the one or more chimps into one or more independent groups, wherein the independent groups comprise at least one of an attacker, a barrier, a chaser, and a driver;
assign in each independent group, an optimal chimp from the one or more chimps;
extract each independent group of the assigned optimal chimp;
update the initiated fitness value of the candidate solution (f), the mean or average fitness value of the population of candidate solutions (m), and the exploration probability (c), of each of the one or more chimps, based on a group strategy of the extracted each independent group;
calculate the acceleration coefficient (a), and a dimensionality (d) of a search space, based on the updated fitness value of the candidate solution (f), the mean or average fitness value of the population of candidate solutions (m), and the exploration probability (c);
determine, if a mutation rate and if a value of an inequality parameter is greater than a pre-defined value or lesser than a pre-defined value, wherein the inequality parameter corresponds to a divergence of the candidate solutions, or a convergence toward the prey based on the value;
update a position of the chimps, when the mutation rate and the value of the inequality parameter are greater than the pre-defined value;
update the position of each chimp, and select a random search agent, when the mutation rate and the value of the inequality parameter are lesser than the pre-defined value;
update the fitness value of a candidate solution (f), the mean or average fitness value of the population of candidate solutions (m), the exploration probability (c), and the acceleration coefficient (a), based on the updated position of the chimps;
evaluate the fitness function of each chimp;
determine if the termination criteria are met for the candidate solution based on the evaluated fitness function, wherein the termination criteria corresponds to at least one of a maximum number of iterations and a desired level of fitness value; and
display on the user interface of the user device, the optimal chimp, when the termination criteria are met, and assign the optimal chimp in each group when the termination criteria are not met.
